(19) United States
(12) Patent Application Publication
(10) Pub. No.: US 2007/0060548 A1
(43) Pub. Date: Mar. 15, 2007

(54) Modulation of Epac, phospholipase Cepsilon, and phospholipase D to treat pain

(75) Inventors: Jon D. Levine, San Francisco, CA (US); Robert O. Messing, Foster City, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/447,607

(22) Filed: Jun. 6, 2006

Related U.S. Application Data
(60) Provisional application No. 60/688,546, filed on Jun. 7, 2005.

(51) Int. Cl. C12Q 1/68; G01N 33/53; A61K 31/685

(52) U.S. Cl. 514/78; 435/6; 435/7.1

(57) ABSTRACT

The present invention provides methods, compositions, and kits useful for reducing pain in a subject by inhibiting Epac, PLCε, and/or PLD. In addition, the invention provides a variety of prescreening and screening methods aimed at identifying agents that reduce pain. Methods of the invention can involve assaying test agent binding to Epac, PLCε, or PLD. Alternatively, test agents can be screened for their ability to alter the level of Epac, PLCε, or PLD polypeptides, polynucleotides, or action.

### CROSS-REFERENCE TO RELATED APPLICATIONS

This application claims priority to and benefit of U.S. Ser. No. 60/688,546, Filed Jun. 7, 2005, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under grant no. NIH DE 008973. The Government may have certain rights in the invention.

### FIELD OF THE INVENTION

This invention pertains to methods of reducing pain based on inhibition of the cAMP-activated guanine exchange factor Epac, phospholipase C-epsilon (PLCε), and/or phospholipase D (PLD), as well as to related pharmaceutical compositions and screening methods.

### BACKGROUND OF THE INVENTION

The cardinal symptom of inflammation is increased sensitivity to mechanical stimuli (mechanical hyperalgesia or tenderness). The underlying intracellular signaling pathways as well as the mechanoreceptors involved remain fragmentary. Nevertheless, one signaling component, the epsilon isoform of PKC, has turned out to be important in nociceptor sensitization caused by inflammation (Khasar et al., 1999b; Numazaki et al., 2002; Sweitzer et al., 2004), peripheral neuropathies such as diabetes (Joseph and Levine, 2003a), chronic alcoholism (Dina et al., 2000), and cancer-chemotherapy (Dina et al., 2001; Joseph and Levine, 2003b), as well as the transition from acute to chronic pain (Aley et al., 2000; Parada et al., 2003a; Parada et al., 2003b). However, a signaling pathway leading to activation of PKCε still remained to be elucidated.

Recent evidence indicated signaling from cAMP to PKC, suggesting the signaling through adenylyl cyclase (AC)/cAMP not to involve PKA but to branch upstream of PKA to activate PKC (Gold et al., 1998; Parada et al., 2005). However, a mechanism to account for signaling from cAMP to PKC, in nociception or other functional context, had yet to be established.

In a non-neuronal cell line cAMP has been shown to activate not only PKA but also the guanine exchange factor, Epac (de Rooij et al., 1998; Kawasaki et al., 1998). Epac in turn activates a newly identified phospholipase, PLCε (Schmidt et al., 2001), and could therefore potentially activate novel PKCs, such as PKCε, through phospholipase produced diacylglycerol (DAG) (Parekh et al., 2000). While Epac's role in activation of MAP-kinases is a matter of intensive ongoing investigation (Enserink et al., 2002; Keiper et al., 2004), Epac was not known to mediate activation of PKCs.

Using the model of epinephrine induced PKCε-mediated hyperalgesia (Khasar et al., 1999b; Parada et al., 2003b), the present work demonstrated that cAMP is upstream of PKCε and that Epac through phospholipases mediates the cAMP-PKC crosstalk, leading to translocation and activation of PKCε and to the establishment of inflammatory pain.

### SUMMARY OF THE INVENTION

In one embodiment, the invention provides a method of reducing pain. The method entails administering to a subject in need thereof, an effective amount of an inhibitor, which is an Epac inhibitor, a phospholipase C-epsilon (PLCε) inhibitor, and/or a phospholipase D (PLD) inhibitor. In particular embodiments, the administration of the inhibitor results in the subject having decreased hyperalgesia, preferably with no significant effect on nociception.

In certain embodiments, the subject suffers from inflammatory pain, which can be either acute or chronic. The inflammatory pain can be due, for example, to: sunburn, arthritis, colitis, carditis, dermatitis, myositis, neuritis, mucositis, urethritis, cystitis, gastritis, pneumonitis, and/or collagen vascular disease.

Alternatively, or in addition, the subject can be one who suffers from neuropathic pain, which can be acute or chronic. The neuropathic pain can be due, for example, to: causalgia, diabetes, collagen vascular disease, trigeminal neuralgia, spinal cord injury, brain stem injury, thalamic pain syndrome, complex regional pain syndrome type I/reflex sympathetic dystrophy, Fabry's syndrome, small fiber neuropathy, cancer, cancer chemotherapy, chronic alcoholism, stroke, abscess, demyelinating disease, viral infection, anti-viral therapy, AIDS, and/or AIDS therapy. Neuropathic pain amenable to treatment according to the method of the invention includes pain due to an agent selected from the group consisting of: trauma, surgery, amputation, toxin, and chemotherapy.

The method of the invention can also be used to treat a subject suffering from a generalized pain disorder, such as, for example, fibromyalgia, irritable bowel syndrome, and a temporomandibular disorder.

An Epac inhibitor useful for reducing pain can, but need not, act directly on Epac. In a particular embodiment, the method includes administering an Epac inhibitor to the subject and additionally administering an analgesic agent that acts by a different mechanism than the Epac inhibitor.

A PLCε inhibitor useful for reducing pain can, but need not, act directly on PLCε. In a particular embodiment, the method includes administering a PLCε inhibitor to the subject and additionally administering an analgesic agent that acts by a different mechanism than the PLCε inhibitor.

A PLD inhibitor useful for reducing pain can, but need not, act directly on PLD. In preferred embodiments, the PLD inhibitor is a selective PLD inhibitor. In a particular embodiment, the method includes administering a PLD inhibitor to the subject and additionally administering an analgesic agent that acts by a different mechanism than the PLD inhibitor.

Any of the inhibitors useful in the method of the invention can also be co-administered with: an inhibitor of protein kinase A (PKA), an inhibitor of cAMP, a nonsteroidal anti-inflammatory drug, a prostaglandin synthesis inhibitor, a local anesthetic, an anticonvulsant, an antidepressant, an opioid receptor agonist, and/or a neuroleptic.

Another aspect of the invention is a pharmaceutical composition. The pharmaceutical composition can include: (a) an Epac inhibitor, a PLCε inhibitor, and/or a PLD inhibitor; and (b) an analgesic agent that acts by a different mechanism than said inhibitor. In another embodiment, the pharmaceutical composition includes: (a) an Epac inhibitor, a PLCε inhibitor, and/or a PLD inhibitor; and (b) one or more of the following agents: an inhibitor of protein kinase A (PKA), an inhibitor of cAMP, a nonsteroidal anti-inflammatory drug, prostaglandin synthesis inhibitor, a local anesthetic, an anticonvulsant, an antidepressant, an opioid receptor agonist, and a neuroleptic.

The invention also provides methods of prescreening and screening for an agent that can reduce pain in a subject. A prescreening method based on polypeptide binding entails: (a) contacting a test agent with one of the following polypeptides: Epac, PLCε, and PLD; (b) determining whether the test agent specifically binds to the polypeptide; and (c) if the test agent specifically binds to the polypeptide, selecting the test agent as a potential analgesic. A prescreening method based on polynucleotide binding entails: (a) contacting a test agent with a polynucleotide encoding one of the following polypeptides: Epac, PLCε, and PLD; (b) determining whether the test agent specifically binds to the polynucleotide; and (c) if the test agent specifically binds to the polynucleotide, selecting the test agent as a potential analgesic. Either prescreening method can additionally include recording any test agent that specifically binds to the polypeptide or the polynucleotide, respectively, in a database of candidate analgesics. In preferred embodiments, the prescreening method is carried out in vitro.

A screening method of the invention entails: (a) contacting a test agent with one of the following polypeptides: Epac, PLCε, and PLD; (b) determining whether the test agent inhibits the polypeptide; and (c) if the test agent inhibits the polypeptide, selecting the test agent as a potential analgesic. The screening method can additionally include recording any test agent that inhibits the polypeptide in a database of candidate analgesics. In particular embodiments, the screening method is carried out in vitro. In one variation of the screening method, (a) a test agent is contacted with a cell that expresses the polypeptide in the absence of test agent, or with a fraction of that cell; (b) the determination of whether the test agent inhibits the polypeptide includes determining the level of the polypeptide or of polynucleotides encoding the polypeptide; and (c) the test agent is selected as a potential analgesic if the level of the polypeptide, or of polynucleotides encoding the polypeptide, is reduced. In another variation of the screening method, (a) a test agent is contacted with a cell that expresses the polypeptide in the absence of test agent, or with a fraction of that cell; (b) the determination of whether the test agent inhibits the polypeptide includes determining the level of an action of the polypeptide; (c) and the test agent is selected as a potential analgesic if the level of polypeptide action is reduced.

In particular embodiments, the screening method of the invention can include combining the selected test agent with a pharmaceutically acceptable carrier and/or measuring the ability of the selected test agent to reduce pain in an animal model.

An in vivo screening method of the invention entails: (a) selecting one of the following inhibitors as a test agent: an Epac inhibitor, a PLCε inhibitor, and a PLD inhibitor; and (b) measuring the ability of the selected test agent to reduce pain in an animal model.

Another aspect of the invention is a kit that includes: (a) one of the following inhibitors in a pharmaceutically acceptable carrier: an Epac inhibitor, a PLCε inhibitor, and a PLD inhibitor; and (b) instructions for carrying out the method of the invention for reducing pain in a subject.

### BRIEF DESCRIPTION OF THE DRAWINGS

FIG. 1: β2-adrenergic receptor (β2-AR) agonist induced translocation of PKCε in cultured DRG-neurons. A) Confocal images of representative untreated (left) versus isoproterenol (1 μM, 30 s) treated (right) cultured DRG neurons. Cultures were fixed after treatment, incubated with the affinity-purified PKCε-specific antiserum SN134 (1:1000) and detected with FITC-coupled donkey anti-rabbit IgG serum (1:200). Insets show enlarged area indicated by the white frame. After isoproterenol treatment a portion of PKCε can be seen to translocate to the plasma membrane. White scale bar equals 20 μm. B) Percentage of neurons demonstrating after 30 s PKCε translocation to the plasma membrane in response to indicated concentrations of isoproterenol. C) Percentage of neurons demonstrating PKCε translocation with isoproterenol (1 μM) stimulation, for indicated timepoints. Filled bars represent cultures treated with 1 μM isoproterenol. Cultures represented by dotted bars were pretreated for 15 min with the β2-AR specific inhibitor ICI 118,551 (50 μM) before stimulation with 1 μM isoproterenol. p<0.01 indicated by **.

FIG. 2: G-protein αs, adenylyl cyclase, and Epac but not PKA are involved in PKCε translocation. A) DRG cultures were pretreated for 15 min with indicated concentrations (1×, 10×, 100×, 1000× the CMIQ-IC50 (0.03-30 μM)) of the PKA-specific inhibitor 4-Cyano-3-methylisoquinoline (CMIQ)(Lu et al., 1996) before stimulation with 1 μM isoproterenol for 30 s. Cultures not treated with either CMIQ or isoproterenol served as negative controls. B) Injection of the PKA inhibitor CMIQ (2.5 μg/2.5 μl) intradermally in rat paws did not change the mechanical paw-withdrawal threshold. Active PKA by injection of its catalytic subunit (PKAcs, 25 Units/2.5 μl) induced robust mechanical hyperalgesia. Preinjection of CMIQ 15 minutes prior to the injection of PKAcs completely blocked the PKA induced hyperalgesia in vivo. C) Cultures were stimulated with activators of, G-protein as (cholera toxin (1 μg/ml), adenylyl cyclase (forskolin, 5 μM), and Epac (CPTOMe 10 μM), for the indicated time. Unstimulated cells served as a negative control, and isoproterenol (1 μM, 30 s) treated cells, as a positive control. P<0.05 is indicated by *, p<0.01 indicated by **.

FIG. 3: β2-AR-induced Epac-mediated translocation of PKCε requires the activity of both, PI—PLC and PLD. A) Percentage of neurons demonstrating PKCε translocation to the plasma membrane after stimulation with isoproterenol (Iso, 1 μM, 30 s). Cultures were pretreated for 15 min with the indicated inhibitors: PC—PLC inhibitor, D-609 (30 μM); PI—PLC inhibitor, U73122 (10 μM); inactive homolog of U73122, U73343 (10 μM); PLD inhibitor, 1-butanol (50 mM); inactive homolog for 1-butanol, 2-butanol (50 mM); PKC kinase inhibitor, Bisindolylmaleimide I (BIM, 100 nM). Concentrations used are roughly 10-times reported IC50 values. B) Percentage of neurons demonstrating PKCε translocation to the plasma membrane after stimulation with the Epac specific activator CPTOMe (10 μM, 90 s). Inhibitors were used as in A). P<0.01 indicated by **.

FIG. 4: In vivo Epac mediates epinephrine-induced hyperalgesia via PI—PLC, PLD and PKCε. A) Injection of epinephrine (0.1 μg in 2.5 μl) and CTPOMe (6.3 μg in 2.5 μl) produce hyperalgesia of similar magnitude, while saline vehicle injection has no effect. CPTOMe-induced sensitization can be completely blocked by the pre-injection of the specific PKCε inhibitory peptide εV1-2 (1 μg in 2.5 μl), 30 min before stimulation with CPTOMe, demonstrating, that also in vivo Epac induces mechanical hyperalgesia through activation of PKCε. B) Epinephrine-induced (filled bars) and CPTOMe-induced (dotted bars) mechanical hyperalgesia can be completely blocked by preinjection of the PI—PLC inhibitor U73122 (2.5 μl, 1 μg/μl) but not its inactive control, U73343 (2.5 μl, 1 μg/μl), 30 min before Epinephrine/CPTOMe stimulation. Likewise, resembling the in vitro data, the injection of the PLD inhibitor, 1-butanol (2.5 μl, 10.9 M) but not its inactive control, 2-butanol, inhibits completely the sensitization through Epinephrine/CPTOMe injection. The inhibitors show no or little effect on saline control injections (open bars). Both phospholipases are therefore also necessary for the mediation of β2-AR-stimulated/Epac/PKCε-mediated mechanical hyperalgesia in vivo. P<0.01 indicated by **.

FIG. 5: PKCε translocation and IB4 double staining. Epifluorescence images of double stained cells tested for PKCε (image A and C, 1:1000 diluted) and IB4 (image B and D, 1:100 diluted). While the cell in the upper row is translocating PKCε to the plasma membrane (A) it shows also clear plasma membrane staining of the 1B4 epitope (C). In contrast, the cell in (C) does not translocate PKCε and is not positive for the 1B4 epitope (D). Insets show enlarged area indicated by the white frame. White bars equal 20 μm.

FIG. 6: β2-AR agonists signal through Epac, PI—PLC and PLD to PKCε, resulting in mechanical hyperalgesia. Schematic of proposed second messenger signaling cascade for β2-AR agonist-induced mechanical hyperalgesia includes G-protein as leading to activation of AC and Epac in the peripheral terminal of the primary afferent nociceptor. PKA is not involved in β2-AR-induced/PKCε-mediated sensitization. Epac leads to the activation of PI—PLC and PLD, the activity of both of which is necessary for the translocation of PKCε in vitro and the onset of Epac/PKCε-mediated hyperalgesia in vivo. As shown earlier, PKCε activation leads to an increase in the TTX—R sodium current (Khasar et al., 1999b), which has a central role in hyperalgesia. Activators/inhibitors used are indicated at their respective level of action on the right of the scheme.

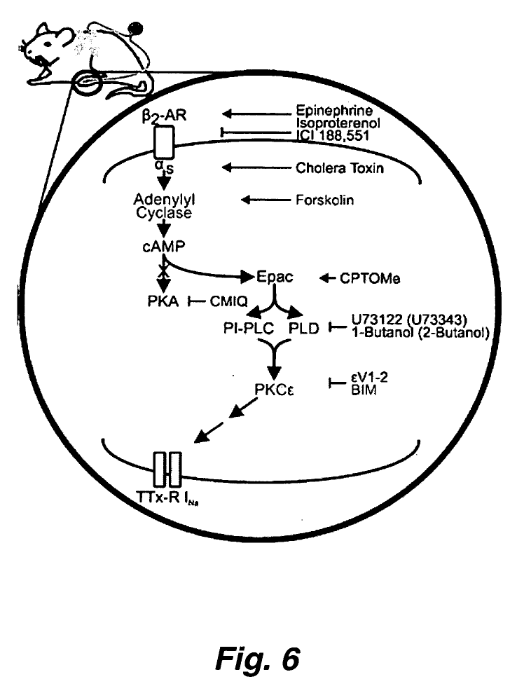

### DETAILED DESCRIPTION

The present invention relates to the discovery that Epac, PLCε, and PLD are mediators of pain, particularly inflammatory and neuropathic pain. Accordingly, the invention provides methods of reducing pain based on inhibiting Epac, PLCε, and/or PLD and related pharmaceutical compositions. In addition, the method provides methods of screening for new agents that can reduce pain in a subject based on screening for agents that bind to, and/or inhibit, Epac, PLCε, or PLD polypeptides or polynucleotides.

Definitions

Terms used in the claims and specification are defined as set forth below unless otherwise specified.

The following terms encompass polypeptides that are identified in Genbank by the following designations, as well as polypeptides that are at least about 70% identical to polypeptides identified in Genbank by these designations: any of family of the cAMP-activated guanine exchange factors for Rap1 (including Epac1 and Epac2), phospholipase C-epsilon (PLCε) (including the splice variants PLCε1a and PLCε1b), and phospholipase D (including PLD1 and PLD2). In alternative embodiments, these terms encompass polypeptides identified in Genbank by these designations and sharing at least about 80, 90, 95, 96, 97, 98, or 99% identity.

A “modulator” of a polypeptide is either an inhibitor or an enhancer of an action or function of the polypeptide.

A “non-selective” modulator of a polypeptide (e.g., PLD) is an agent that modulates other members of the same family of polypeptides (e.g., other phospholipases) at the concentrations typically employed for modulation of the particular polypeptide.

A “selective” modulator of a polypeptide significantly modulates the particular polypeptide at a concentration at which other members of the same family of polypeptides are not significantly modulated. Thus, a modulator can be selective for, e.g., PLD versus PLC.

A modulator “acts directly on” a polypeptide when the modulator exerts its action by interacting directly with the polypeptide.

A modulator “acts indirectly on” a polypeptide when the modulator exerts its action by interacting with a molecule other than the polypeptide, which interaction results in modulation of an action or function of the polypeptide.

An “inhibitor” or “antagonist” of a polypeptide is an agent that reduces, by any mechanism, any action or function of the polypeptide, as compared to that observed in the absence (or presence of a smaller amount) of the agent. An inhibitor of a polypeptide can affect: (1) the expression, mRNA stability, protein trafficking, modification (e.g., phosphorylation), or degradation of a polypeptide, or (2) one or more of the normal action or functions of the polypeptide. An inhibitor of a polypeptide can be non-selective or selective. Preferred inhibitors (antagonists) are generally small molecules that act directly on, and are selective for, the target polypeptide.

An “enhancer” or “activator” is an agent that increases, by any mechanism, any polypeptide action or function, as compared to that observed in the absence (or presence of a smaller amount) of the agent. An enhancer of a polypeptide can affect: (1) the expression, mRNA stability, protein trafficking, modification (e.g., phosphorylation), or degradation of a polypeptide, or (2) one or more of the normal actions or functions of the polypeptide. An enhancer of a polypeptide can be non-selective or selective. Preferred enhancers (activators) are generally small molecules that act directly on, and are selective for, the target polypeptide.

The terms “polypeptide” and “protein” are used interchangeably herein to refer a polymer of amino acids, and unless otherwise limited, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3- (and 4-) hydoxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CHl by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH—VL heterodimer which may be expressed from a nucleic acid including VH— and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, F light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778).

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH7.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

The phrases “an effective amount” and “an amount sufficient to” refer to amounts of a biologically active agent that produce an intended biological activity.

The term “co-administer,” when used in reference to the administration of Epac and/or PLD inhibitors and other analgesic agents indicates that the inhibitors are administered so that there is at least some chronological overlap in their physiological activity on the subject. Thus, an Epac and/or PLD inhibitor can be administered simultaneously and/or sequentially with another analgesic. In sequential administration, there may even be some substantial delay (e.g., minutes or even hours or days) before administration of the second agent as long as the first administered agent is exerting some physiological effect on the organism when the second administered agent is administered or becomes active in the subject.

The term “reducing pain,” as used herein, refers to decreasing the level of pain a subject perceives relative to the level of pain the subject would have perceived were it not for the intervention. Where the subject is a person, the level of pain the person perceives can be assessed by asking him or her to describe the pain or compare it to other painful experiences. Alternatively, pain levels can be determined by measuring the subject's physical responses to the pain, such as the release of stress-related factors or the activity of pain-transducing nerves in the peripheral nervous system or the CNS. One can also determine pain levels by measuring the amount of a well-characterized analgesic required for a person to report that no pain is present or for a subject to stop exhibiting symptoms of pain. A reduction in pain can also be measured as an increase in the threshold at which a subject experiences a given stimulus as painful. In certain embodiments, a reduction in pain is achieved by decreasing “hyperalgesia,” the heightened sensitivity to a noxious stimulus, and such inhibition can occur without impairing nociception, the subject's normal sensitivity to a “noxious” stimulus.

As used with reference to pain reduction, “a subject in need thereof” refers to an animal or person, preferably a person, expected to experience pain in the near future. Such animal or person may have a ongoing condition that is causing pain currently and is likely to continue to cause pain. Alternatively, the animal or person has been, is, or will be enduring a procedure or event that usually has painful consequences. Chronic painful conditions such as diabetic neuropathic hyperalgesia and collagen vascular diseases are examples of the first type; dental work, particularly that accompanied by inflammation or nerve damage, and toxin exposure (including exposure to chemotherapeutic agents) are examples of the latter type.

“Inflammatory pain” refers to pain arising from inflammation. Inflammatory pain often manifests as increased sensitivity to mechanical stimuli (mechanical hyperalgesia or tenderness).

“Neuropathic pain” refers to pain arising from conditions or events that result in nerve damage. “Neuropathy” refers to a disease process resulting in damage to nerves. “Causalgia” denotes a state of chronic pain following nerve injury. “Allodynia” refers to a condition in which a person experiences pain in response to a normally nonpainful stimulus, such as a gentle touch.

As used herein, the term “generalized pain disorder” refers to a group of idiopathic pain syndromes (e.g., fibromyalgia, irritable bowel syndrome, and temporomandibular disorders), for which the pathogenic mechanism is currently unknown, characterized by diffuse or generalized pain, and for which a diagnosis of inflammation or neuropathy as the direct cause of pain is excluded.

An “analgesic agent” refers to a molecule or combination of molecules that causes a reduction in pain. An analgesic agent employs a mechanism of action other than inhibition of Epac, PLCε, or PLD when its mechanism of action does not involve direct (via electrostatic or chemical interactions) interaction with, and reduction in the action or function of Epac, PLCε, or PLD or any intracellular molecule in the Epac/PLCε/PLD pathway.

A “neuroleptic” refers to a class of tranquilizing drugs, used to treat psychotic conditions, that modulate neurotransmitter activity in the central nervous system and can act by modulating acetylcholine, dopamine, norepinephrine, serotonin, or γ-aminobutyric acid (GABA) transmission.

The difference between “acute” and “chronic” pain is one of timing: acute pain is experienced soon (e.g., generally within about 48 hours, more typically within about 24 hours, and most typically within about 12 hours) after the occurrence of the event (such as inflammation or nerve injury) that led to such pain. By contrast, there is a significant time lag between the experience of chronic pain and the occurrence of the event that led to such pain. Such time lag is generally at least about 48 hours after such event, more typically at least about 96 hours after such event, and most typically at least about one week after such event.

A “test agent” is any agent that can be screened in the prescreening or screening assays of the invention. The test agent can be any suitable composition, including a small molecule, peptide, or polypeptide.

Method of Reducing Pain

A. In General

The invention provides a method of reducing pain. The method entails administering to a subject in need of pain reduction, an effective amount of an inhibitor of Epac, phospholipase C-epsilon (PLCε), and/or phospholipase D (PLD).

The subject of the method can be any individual that expresses Epac, phospholipase C-epsilon (PLCε), and/or PLD and has a measurable response to pain. Examples of suitable subjects include research animals, such as mice, rats, guinea pigs, rabbits, cats, dogs, as well as monkeys and other primates, and humans. In a particularly useful embodiment, the subject suffers from hyperalgesia, and administration of an inhibitor according to the invention reduce hyperalgesia, preferably without affecting nociception. In this instance, a subject treated with such an inhibitor will have relief from excessive pain, e.g., stemming from innocuous stimuli while still being able to sense pain normally in response to noxious stimuli.

In one embodiment, the subject suffers from inflammatory pain, which may be acute or chronic. Examples of inflammatory pain amendable to treatment by inhibiting Epac, PLCε, and/or PLD include pain due to sunburn, arthritis, colitis, carditis, dermatitis, myositis, neuritis, mucositis, urethritis, cystitis, gastritis, pneumonitis,and collagen vascular disease.

In another embodiment, the subject suffers from neuropathic pain, which also may be acute or chronic. Examples of neuropathic pain amendable to treatment by inhibiting Epac, PLCε, and/or PLD include pain due to conditions such as, e.g., causalgia, diabetes, collagen vascular disease, trigeminal neuralgia, spinal cord injury, brain stem injury, thalamic pain syndrome, complex regional pain syndrome type I/reflex sympathetic dystrophy, Fabry's syndrome, small fiber neuropathy, cancer, cancer chemotherapy, chronic alcoholism, stroke, abscess, demyelinating disease, viral infection, anti-viral therapy, AIDS, and AIDS therapy. Inflammatory pain arising from, e.g., trauma, surgery, amputation, toxin, and/or chemotherapy can also be treated using the inhibitors of the invention.

In particular embodiments, the subject suffers from a generalized pain disorder, such as, e.g., fibromyalgia, irritable bowel syndrome, and/or temporomandibular disorders.

The method of the invention entails inhibiting Epac, PLCε, and/or PLD to a degree sufficient to reduce pain experiences by the subject. In various embodiments, Epac, PLCε, and/or PLD is inhibited by at least about 10, 20, 30, 40, 50, 60, 70, 80, 90, and 95 percent, as determined by any suitable measure of Epac, PLCε, and/or PLD inhibition (such as, for example, any of the assays described herein).

1. Inhibition of Epac

Any kind of Epac inhibitor that is tolerated by the subject can be employed in the method of the invention. Thus, the inhibitor can be a polypeptide (such as, e.g., an anti-Epac antibody), a polynucleotide (e.g., an inhibitory RNA or a polynucleotide that encodes an inhibitory polypeptide), or a small molecule. In particular embodiments, when the inhibitor is a polynucleotide-encoded inhibitory polypeptide, the polynucleotide is introduced into the subject's cells, where the encoded polypeptide is expressed in an amount sufficient to inhibit Epac.

Inhibition of Epac can be achieved by any available means, e.g.: (1) inhibition of the expression, mRNA stability, protein trafficking, or modification of Epac; (2) stimulation of degradation of Epac; or (3) inhibition of one or more of the normal functions of Epac, such as guanine exchange. In preferred embodiments, the Epac inhibitor acts directly on Epac.

In one embodiment, Epac inhibition is achieved by reducing the level of Epac polypeptides in any target tissue in which the Epac/PLCε/PLD pathway is active. This pathway is active, for example, in neurons of the central nervous system, particularly in dorsal root ganglion neurons, and more particularly in isolectin B4-positive (IB4(+)) nociceptors. Epac levels can be reduced using, e.g., antisense or RNA interference (RNAᵢ) techniques.

In other embodiments, the Epac inhibitor can be, e.g., a peptide or a small molecule identified through a screening assay of the invention, which are described below.

2. Inhibition of PLCε

PLCε can be inhibited according to the method of the invention using any kind of PLCε inhibitor that is tolerated by the subject. Thus, the inhibitor can be a polypeptide (such as, e.g., an anti-PLCε antibody), a polynucleotide (e.g., an inhibitory RNA or one that encodes an inhibitory polypeptide), or a small molecule. In particular embodiments, when the inhibitor is a polynucleotide-encoded inhibitory polypeptide, the polynucleotide is introduced into the subject's cells, where the encoded polypeptide is expressed in an amount sufficient to inhibit PLD.

Inhibition of the PLCε can be achieved by any available means, e.g.: (1) inhibition of the expression, mRNA stability, protein trafficking, or modification of PLCε; (2) stimulation of degradation of PLCε; or (3) inhibition of one or more of the normal functions of PLCε, such as, e.g., hydrolysis of phospholipids (primarily phosphotidylinositol) to generate inositol triphosphate, along with diacylglycerol (DAG). In preferred embodiments, the PLCε inhibitor acts directly on PLCε.

In one embodiment, PLCε inhibition is achieved by reducing the level of PLCε in any target tissue in which the Epac/PLCε/PLD pathway is active, such as, for example, in neurons of the central nervous system, particularly in dorsal root ganglion neurons, and more particularly in isolectin B4-positive (IB4(+)) nociceptors. PLCε levels can be reduced using, e.g., antisense or RNA interference (RNAᵢ) techniques.

In preferred embodiments, the PLCε inhibitor can be, e.g., a peptide or a small molecule. A number of peptide or small-molecule PLC inhibitors have been described, including the PI—PLC inhibitor U-73122 (commercially available from Sigma), ET-18OCH3 (Siese, A., et al., Scand J Immunol. February 1999;49(2): 139-48), and neomycin. The selection of suitable PLCε inhibitor for a particular application is within the level of skill in the art.

The PLCε inhibitor can be non-selective or selective for PLCε. Preferred embodiments employ a selective PLCε inhibitor.

3. Inhibition of PLD

PLD can be inhibited according to the method of the invention using any kind of PLD inhibitor that is tolerated by the subject. Thus, the inhibitor can be a polypeptide (such as, e.g., an anti-PLD antibody), a polynucleotide (e.g., an inhibitory RNA or one that encodes an inhibitory polypeptide), or a small molecule. In particular embodiments, when the inhibitor is a polynucleotide-encoded inhibitory polypeptide, the polynucleotide is introduced into the subject's cells, where the encoded polypeptide is expressed in an amount sufficient to inhibit PLD.

Inhibition of PLD can be achieved by any available means, e.g.: (1) inhibition of the expression, mRNA stability, protein trafficking, or modification of PLD; (2) stimulation of degradation of PLD; or (3) inhibition of one or more of the normal functions of PLD, such as, e.g., hydrolysis of phospholipids (primarily phosphatidylcholine) to generate phosphatidic acid, which is converted to diacylglycerol (DAG). In preferred embodiments, the PLD inhibitor acts directly on PLD.

In one embodiment, PLD inhibition is achieved by reducing the level of PLD in any target tissue in which the Epac/PLCε/PLD pathway is active, such as, for example, in neurons of the central nervous system, particularly in dorsal root ganglion neurons, and more particularly in isolectin B4-positive (IB4(+)) nociceptors. PLD levels can be reduced using, e.g., antisense or RNA interference (RNAᵢ) techniques.

In preferred embodiments, the PLD inhibitor can be, e.g., a peptide or a small molecule. A number of small-molecule PLD inhibitors have been described, including ethylenediaminetetraacetic acid tripotassium salt dehydrate (Sigma), ethylene glycol-bis(2-aminoethylether)-N,N,N′,N′-tetraacetic acid (Sigma), C(2)-ceramide (Nishimaru, K., et al., J. Pharmacol. Sci. (2003) 92:196-202), and 1-butanol (Jackson, T. C., et al., J. Pharm. Exp. Therapeutics Fast Forward (2004) DOI: 10.1124/jpet.103.063081). The selection of suitable PLD inhibitor for a particular application is within the level of skill in the art.

The PLD inhibitor can be non-selective or selective for PLD. Preferred embodiments employ a selective PLD inhibitor.

4. Methods of Inhibiting Epac, PLCε, and/or PLD

A variety of techniques are available that permit the inhibition of any gene or protein of interest. Any of these can be employed in the method of the invention, including the five exemplary techniques are described below.

a. Antisense Methods

Epac, PLCε, and/or PLD gene expression can be reduced or entirely blocked by the use of antisense molecules. An “antisense sequence or antisense polynucleotide” is a polynucleotide that is complementary to the Epac, PLCε, and/or PLD coding mRNA sequence or a subsequence thereof. Binding of the antisense molecule to the Epac, PLCε, and/or PLD mRNA interferes with normal translation of the encoded polypeptide.

Thus, in particular embodiments, the invention provides antisense molecules useful for inhibiting Epac, PLCε, and/or PLD. Suitable antisense molecules include oligonucleotides and oligonucleotide analogs that are hybridizable with Epac, PLCε, and/or PLD mRNA. The oligonucleotides and oligonucleotide analogs are able to inhibit the function of the RNA, either its translation into protein, its translocation into the cytoplasm, or any other activity necessary to its overall biological function. The failure of the mRNA to perform all or part of its normal functions results in a partial or complete inhibition of expression of Epac, PLCε, and/or PLD polypeptides.

Oligonucleotides useful in the antisense methods of the invention include polynucleotides formed from naturally-occurring bases and/or cyclofuranosyl groups joined by native phosphodiester bonds. The term “oligonucleotide” encompasses moieties that function similarly to oligonucleotides, but that have non-naturally occurring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short-chain alkyl or cycloalkyl structures. In accordance with other preferred embodiments, the phosphodiester bonds are substituted with structures that are, at once, substantially non-ionic and non-chiral, or with structures which are chiral and enantiomerically specific. Persons of ordinary skill in the art will be able to select other linkages for use in the practice of the invention.

In an exemplary embodiment, the internucleotide phosphodiester linkage is replaced with a peptide linkage. Such peptide polynucleotides tend to show improved stability, penetrate the cell more easily, and show enhanced affinity for their target. Methods of making peptide polynucleotides are known to those of skill in the art (see, e.g., U.S. Pat. Nos: 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides useful in the antisense methods of the invention may also include one or more modified base forms. Thus, purines and pyrimidines other than those normally found in nature may be employed. Similarly, the furanosyl portions of the nucleotide subunits may also be modified, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are: OH, SH, SCH₃, F, OCH₃, OCN, O(CH₂)[n]NH₂ or O(CH₂)[n]CH₃, where n is from 1 to about 10, and other substituents having similar properties.

All such analogs can be used in the antisense methods of the invention so long as the analogs function effectively to hybridize with Epac, PLCε, and/or PLD mRNA and inhibit the function of that RNA.

Antisense oligonucleotides in accordance with this invention preferably comprise from about 3 to about 50 subunits (i.e., bases in unmodified polynucleotides). It is more preferred that such oligonucleotides and analogs comprise from about 8 to about 25 subunits and still more preferred to have from about 12 to about 20 subunits. The oligonucleotides used in accordance with this invention can be conveniently and routinely made through the well-known technique of solid phase synthesis. Equipment for such synthesis is sold by several vendors (e.g. Applied Biosystems).

Antisense oligonucleotides of the-invention can be synthesized, formulated, and administered to cells, tissues, or organisms in accordance with standard practice. General considerations with respect to administration and dose are discussed below. Formulations containing at least one component that facilitates entry of a polynucleotide into a cell are discussed below with respect to compositions containing polynucleotide inhibitors of Epac, PLCε, and/or PLD. Those of skill in the art will readily appreciate that this discussion is equally applicable to antisense oligonucleotides, catalytic RNAs and DNAs, and double-stranded RNAs used in RNAi. Similarly, those of skill in the art understand that antisense oligonucleotides can be introduced into host cells as described below for polynucleotide inhibitors, generally.

b. Catalytic RNAs and DNAs

(1) Ribozymes

In another approach, Epac, PLCε, and/or PLD expression can be inhibited by the use of ribozymes. As used herein, “ribozymes” include RNA molecules that contain antisense sequences for specific recognition, and an RNA-cleaving enzymatic activity. The catalytic strand cleaves a specific site in a target (Epac, PLCε, and/or PLD) RNA, preferably at greater than stoichiometric concentration. The ribozymes of the invention typically consist of RNA, but such ribozymes may also be composed of polynucleotide molecules comprising chimeric polynucleotide sequences (such as DNA/RNA sequences) and/or polynucleotide analogs (e.g., phosphorothioates).

Accordingly, one aspect of the present invention includes ribozymes have the ability to inhibit Epac, PLCε, and/or PLD expression. Such ribozymes may, e.g., be in the form of a “hammerhead” (for example, as described by Forster and Symons (1987) Cell 48: 211-220; Haseloff and Gerlach (1988) Nature 328: 596-600; Walbot and Bruening (1988) Nature 334: 196; Haseloff and Gerlach (1988) Nature 334: 585); Rossi et al. (1991) Pharmac. Ther. 50: 245-254) or a “hairpin” (see, e.g., U.S. Pat. No. 5,254,678 and Hampel et al., European Patent Publication No. 0 360 257, published Mar. 26, 1990; Hampel et al. (1990) Nucl. Acids Res. 18: 299-304), and have the ability to specifically target and cleave and Epac, PLCε, and/or PLD polynucleotides.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO:1). Suitable Epac, PLCε, and/or PLD recognition or target sequences for hairpin ribozymes can be readily determined from the Epac, PLCε, and/or PLD sequence.

The sequence requirement at the cleavage site for the hammerhead ribozyme is any RNA sequence consisting of NUX (where N is any of G, U, C, or A and X represents C, U, or A). Accordingly, the same target within the hairpin leader sequence, GUC, is useful for the hammerhead ribozyme. The additional nucleotides of the hammerhead ribozyme or hairpin ribozyme are determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an 8-base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. Preferred ribozymes of the invention hybridize efficiently to target sequences at physiological temperatures, making them particularly well suited for use in vivo.

Ribozymes, as well as DNA encoding such ribozymes, and other suitable polynucleotide molecules can be chemically synthesized using methods well known in the art for the synthesis of polynucleotide molecules. Alternatively, Promega, Madison, Wis., USA, provides a series of protocols suitable for the production of RNA molecules such as ribozymes. The ribozymes also can be prepared from a DNA molecule or other polynucleotide molecule (which, upon transcription, yields an RNA molecule) operably linked to an RNA polymerase promoter, e.g., the promoter for T7 RNA polymerase or SP6 RNA polymerase (e.g., a vector that provides an intiation site and template for transcription). Accordingly, also provided by this invention are polynucleotide molecules, e.g., DNA or cDNA, coding for the ribozymes of this invention. When the vector also contains an RNA polymerase promoter operably linked to the polynucleotide molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the corresponding phosphothio analog for use in liposome delivery systems. This modification also renders the ribozyme resistant to endonuclease activity.

Ribozymes, or polynucleotides encoding them (e.g., DNA vectors), can be formulated, and administered to cells, tissues, or organisms in accordance with standard practice. General considerations with respect to administration and dose are discussed below. Formulations containing at least one component that facilitates entry of a polynucleotide into a cell are discussed below with respect to compositions containing polynucleotide inhibitors of Epac, PLCε, and/or PLD. Those of skill in the art will readily appreciate that ribozymes, or polynucleotides encoding them, can be introduced into host cells as described below for polynucleotide inhibitors, generally.

When a vector containing an encoded ribozyme linked to a promoter for RNA transcription, the RNA can be produced in the host cell when the host cell is grown under suitable conditions favoring transcription of the vector. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W. H. Freeman and Company, New York.

To produce ribozymes in vivo utilizing such vectors, the nucleotide sequence endoding the ribozyme is preferably operably linked to a strong promoter such as the lac, SV40 late, SV40 early, or lambda promoters.

(2) Catalytic DNA

In a manner analogous to ribozymes, DNA molecules are also capable of catalytic (e.g. nuclease) activity. For example, highly catalytic species have been developed by directed evolution and selection. Beginning with a population of 10¹⁴ DNAs containing 50 random nucleotides, successive rounds of selective amplification enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is composed of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹ min⁻¹ under multiple turnover conditions, exceeding that of any other known polynucleotide enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to Epac, PLCε, and/or PLD mRNA and can be used in essentially the same manner as described above for Epac, PLCε, and/or PLD ribozymes.

c. RNAi Methods

Post-transcriptional gene silencing (PTGS) or RNA interference (RNAi) refers to a mechanism by which double-stranded (sense strand) RNA (dsRNA) specifically blocks expression of its homologous gene when injected, or otherwise introduced into cells. This approach is based on the observation that injection of antisense or sense RNA strands into C. elegans cells resulted in gene-specific inactivation (Guo and Kempheus (1995) Cell 81: 611-620). While gene inactivation by the antisense strand was expected, gene silencing by the sense strand was unexpected. Surprisingly, it was determined that the gene-specific inactivation was actually due to trace amounts of contaminating dsRNA (Fire et al. (1998) Nature 391: 806-811).

Since then, this mode of post-transcriptional gene silencing has been demonstrated in a wide variety of organisms: plants, flies, trypanosomes, planaria, hydra, zebrafish, and mice (Zamore et al. (2000) Cell 101: 25-33; Gura (2000) Nature 404: 804-808). RNAi activity has been associated with functions as disparate as transposon-silencing, anti-viral defense mechanisms, and gene regulation (Grant (1999) Cell 96: 303-306).

By injecting dsRNA into tissues, one can inactivate specific genes not only in those tissues, but also during various stages of development. This is in contrast to tissue-specific knockouts or tissue-specific dominant-negative gene expression, which do not allow for gene silencing during various stages of the developmental process (Gura (2000) Nature 404:804-808).

dsRNA can be formulated, and administered to cells, tissues, or organisms in accordance with standard practice. General considerations with respect to administration and dose are discussed below, as are formulations containing at least one component that facilitates entry of a polynucleotide into a cell (discussed below with respect to compositions containing polynucleotide inhibitors of Epac, PLCε, and/or PLD). Those of skill in the art will readily appreciate that dsRNA can be introduced into host cells as described below for polynucleotide inhibitors, generally. Additionally, dsRNA can be synthesized using one or more vectors designed to transcribe the two complementary RNA strands that hybridize to form the dsRNA (see the discussion of this approach with respect to ribozymes, above). These may be introduced into host cells using any of the techniques described herein or known in the art for this purpose.

After introduction into cells, it has been shown that dsRNA is cleaved by a nuclease into 21-23-nucleotide fragments. These fragments, in turn, target the homologous region of their corresponding mRNA, hybridize, and result in a double-stranded substrate for a nuclease that degrades it into fragments of the same size (Hammond et al. (2000) Nature 404:293-298; Zamore et al. (2000) Cell 101:25-33).

d. “Knock-out” Methods

In another approach, Epac, PLCε, and/or PLD can be inhibited simply by “knocking out” the Epac, PLCε, and/or PLD gene, respectively. Typically, this is accomplished by disrupting the Epac, PLCε, and/or PLD gene, the promoter regulating the gene or sequences between the promoter and the gene. Such disruption can be specifically directed to Epac, PLCε, and/or PLD by homologous recombination where a “knockout construct” contains flanking sequences complementary to the domain to which the construct is targeted. Insertion of the knockout construct (e.g., into the Epac, PLCε, and/or PLD gene) results in disruption of that gene. The phrases “disruption of the gene” and “gene disruption” refer to insertion of a nucleic acid sequence into one region of the native DNA sequence (usually one or more exons) and/or the promoter region of a gene so as to reduce or prevent expression of that gene in the cell, as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The genomic DNA sequence to be used in producing the knockout construct is digested with a particular restriction enzyme selected to cut at a location(s) such that a new DNA sequence encoding, e.g., a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable; however, typically it is an antibiotic resistance gene or other gene whose expression or presence in the genome can easily be detected. The marker gene is usually operably linked to its own promoter or to another strong promoter from any source that will be active, or can easily be activated, in the cell into which it is introducied; however, the marker gene need not be linked to its own promoter as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene including, but not limited to, neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement).

The resulting knockout constructs can be delivered to cells in vivo using gene therapy delivery vehicles (e.g., retroviruses, liposomes, lipids, dendrimers, etc.). Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395.

The use of homologous recombination to alter expression of endogenous genes is also described in detail in U.S. Pat. No. 5,272,071, WO 91/09955, WO 93/09222, WO 96/29411, WO 95/31560, and WO 91/12650.

Although embryonic stem (ES) cells can be employed to produce knockout animals, ES cells are not required. In various embodiments, knockout animals can be produced using methods of somatic cell nuclear transfer. In preferred embodiments using such an approach, a somatic cell is obtained from the species in which the Epac, PLCε, and/or PLD gene is to be knocked out. The cell is transfected with a construct that introduces a disruption in the Epac, PLCε, and/or PLD gene (e.g., via homologous recombination). Cells harboring a knocked out Epac, PLCε, and/or PLD gene are selected, e.g., by selecting for expression of a marker encoded by a marker gene used to disrupt the native gene. The nucleus of cells harboring the knockout is then placed in an unfertilized enucleated egg (e.g., eggs from which the natural nuclei have been removed by microsurgery). Once the transfer is complete, the recipient eggs contain a complete set of genes, just as they would if they had been fertilized by sperm. The eggs are then cultured for a period before being implanted into a host mammal (of the same species that provided the egg) where they are carried to term, culminating in the birth of a transgenic animal comprising a nucleic acid construct containing one or more disrupted Epac, PLCε, and/or PLD gene.

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Campbell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrobin III gene by somatic cell nuclear transfer has been reported (Baguisi et al. (1999) Nature Biotechnology 17: 456-461).

Somatic cell nuclear transfer simplifies transgenic procedures by employing a differentiated cell source that can be clonally propagated. This eliminates the need to maintain the cells in an undifferentiated state, thus, genetic modifications, both random integration and gene targeting, are more easily accomplished. Also, by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

e. Intrabodies

In still another embodiment, Epac, PLCε, and/or PLD expression/activity can be inhibited by introducing a nucleic acid construct that expresses an intrabody into the target cells. An intrabody is an intracellular antibody, in this case, capable of recognizing and binding to an Epac, PLCε, and/or PLD polypeptide. The intrabody is expressed by an “antibody cassette” containing: (1) a sufficient number of nucleotides encoding the portion of an antibody capable of binding to the target (Epac, PLCε, and/or PLD polypeptide) operably linked to (2) a promoter that will permit expression of the antibody in the cell(s) of interest. The construct encoding the intrabody is delivered to the cell where the antibody is expressed intracellularly and binds to the target Epac, PLCε, and/or PLD, thereby disrupting the target from its normal action.

In a preferred embodiment, the “intrabody gene” of the antibody cassette includes a cDNA encoding heavy chain variable (V_{H}) and light chain variable (V_{L}) domains of an antibody which can be connected at the DNA level by an appropriate oligonucleotide linker, which on translation, forms a single peptide (referred to as a single chain variable fragment, “sFv”) capable of binding to a target such as an Epac, PLCε, and/or PLD protein. The intrabody gene preferably does not encode an operable secretory sequence, and thus the expressed antibody remains within the cell.

Anti-Epac, PLCε, and/or PLD antibodies suitable for use/expression as intrabodies in the methods of this invention can be readily produced by a variety of methods. Such methods include, but are not limited to, traditional methods of raising polyclonal antibodies, which can be modified to form single chain antibodies, or screening of, e.g., phage display libraries to select for antibodies showing high specificity and/or avidity for Epac, PLCε, and/or PLD.

The antibody cassette is delivered to the cell by any means suitable for introducing polynucleotides into cells. A preferred delivery system is described in U.S. Pat. No. 6,004,940. Methods of making and using intrabodies are described in detail in U.S. Pat. Nos. 6,072,036, 6,004,940, and 5,965,371.

5. Co-Administration of Inhibitors with Other Agents

In a particular embodiment of the method, the Epac, PLCε, and/or PLD inhibitor is co-administered with an analgesic agent that acts by a different mechanism than the inhibitor(s) In a variation of this embodiment, the analgesic agent acts by modulating a different signaling pathway, such as, for example, the protein kinase A pathway. Examples of such agents include nitric oxide, MAPKs (ERK1/2, JNK), cermide, Ca²⁺, NaV1.8, TRPV1. This embodiment is useful, for example, to produce a greater overall reduction in pain, in terms of potency and/or duration of effect, to broaden the spectrum of different types pain amenable to treatment using this method, and/or to reduce the dose of inhibitor(s) and/or analgesics necessary to achieve the desired effect. In this embodiment, the amount of analgesic administered is sufficient to produce analgesia in the subject when co-administered with the selected Epac, PLCε, and/or PLD inhibitor.

Any kind of analgesic that is tolerated by the subject can be employed in the method of the invention. Examples include inhibitors of protein kinase A (PKA), inhibitors of cAMP, nonsteroidal anti-inflammatory drugs (e.g., acetominophen), prostaglandin synthesis inhibitors, local anesthetics, and opioid receptor agonists.

Other agents that are not analgesics, but that may be useful in treating conditions accompanied by significant pain include anticonvulsants, antidepressants, and neuroleptics.

Inhibitors of Epac, PLCε, and/or PLC, together with analgesics or other agents can be co-administered by simultaneous administration or sequential administration. In the case of sequential administration, the first administered agent must be exerting some physiological effect on the organism when the second administered agent is administered or becomes active in the organism.

B. Compositions

For research and therapeutic applications, an Epac, PLC8, or PLD inhibitor is generally formulated to deliver inhibitor to a target site in an amount sufficient to inhibit the Epac, PLCε, or PLD at that site. An analgesic or other agent can, optionally, be included in the inhibitor composition to deliver an effective amount to its target site.

In a particular embodiment of the method, the Epac, PLCε, and/or PLD inhibitor(s) is formulated with an analgesic agent that acts by a different mechanism than the inhibitor(s) In a variation of this embodiment, the analgesic agent acts by modulating a different signaling pathway, such as, for example, the protein kinase A pathway. Examples of such agents include nitric oxide, MAPKs (ERK1/2, JNK), cermide, Ca²⁺, NaV1.8, TRPV1.

Any kind of analgesic that is tolerated by the subject can be employed in the method of the invention. Examples include inhibitors of protein kinase A (PKA), inhibitors of cAMP, nonsteroidal anti-inflammatory drugs, prostaglandin synthesis inhibitors, local anesthetics, and opioid receptor agonists, as discussed above.

Other agents that are not analgesics, but that may be useful in treating conditions accompanied by significant pain include anticonvulsants, antidepressants, and neuroleptics, as discussed above. Accordingly, such agents may also, optionally, be included in the inhibitor compositions of the invention.

Inhibitor compositions according to the invention optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Particular embodiments include sustained-release pharmaceutical compositions. An exemplary sustained-release composition has a semipermeable matrix of a solid hydrophobic polymer to which the inhibitor is attached or in which the inhibitor is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are typically in the form of shaped articles, such as films, or microcapsules.

Where the inhibitor is a polypeptide, exemplary sustained release compositions include the polypeptide attached, typically via ε-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” protein retains the desired function(s).

In another embodiment, a sustained-release composition includes a liposomally entrapped inhibitor. Liposomes are small vesicles composed of various types of lipids, phospholipids, and/or surfactants. These components are typically arranged in a bilayer formation, similar to the lipid arrangement of biological membranes. Liposomes containing inhibitors according to the invention are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

In particular embodiments, the methods of the invention employ pharmaceutical compositions containing a polynucleotide inhibitor or a polynucleotide encoding a polypeptide inhibitor of Epac, PLCε, and/or PLD. Such compositions optionally include other components, as for example, a storage solution, such as a suitable buffer, e.g., a physiological buffer. In a preferred embodiment, the composition is a pharmaceutical composition and the other component is a pharmaceutically acceptable carrier, as described above.

Preferably, compositions containing polynucleotides useful in the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with polynucleotides useful in the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC).

In another embodiment, polynucleotides are complexed to dendrimers, which can be used to introduce polynucleotides into cells. Dendrimer polycations are three-dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides useful in the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides.

C. Administration

Pharmaceutical compositions according to the invention are generally administered according to known methods for administering small-molecule drugs, as well as therapeutic polypeptides, peptides, and polynucleotides them. Suitable routes of administration include, for example, topical, intravenous, intraperitoneal, intracerebral, intraventricular, intramuscular, intraocular, intraarterial, or intralesional routes. Pharmaceutical compositions of the invention can be administered continuously by infusion, by bolus injection, or, where the compositions are sustained-release preparations, by methods appropriate for the particular preparation.

In certain embodiments, the compositions are delivered through the skin using a conventional transdermal drug delivery system, i.e., a transdermal “patch” wherein the composition is typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of a selected composition that is ultimately available for delivery to the surface of the skin. Thus, for example, the reservoir may include the composition in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the reservoir and skin contact adhesive are present as separate and distinct layers, with the adhesive underlying the reservoir which, in this case, may be either a polymeric matrix as described above or a liquid or hydrogel reservoir, or it may take some other form. The backing layer in these laminates, which serves as the upper surface of the device, preferably functions as a primary structural element of the patch and provides the device with much of its flexibility. The material selected for the backing layer is preferably substantially impermeable to the selected composition and any other materials that are present.

Transdermal patches according to the invention can include a rate-limiting patch membrane. The size of the patch and or the rate-limiting membrane can be chosen to deliver the transdermal flux rates desired. A release liner, such as a polyester release liner, can also be provided to cover the adhesive layer prior to application of the patch to the skin as is conventional in the art. This patch assembly can be packaged in an aluminum foil or other suitable pouch, again, as is conventional in the art.

In other embodiments, the compositions of the invention are administered in implantable depot formulations. A wide variety of approaches to designing depot formulations that provide sustained release of an active agent are known and are suitable for use in the invention. Generally, the components of such formulations are biocompatible and may be biodegradable. Biocompatible polymeric materials have been used extensively in therapeutic drug delivery and medical implant applications to effect a localized and sustained release. See Leong et al., “Polymeric Controlled Drug Delivery”, Advanced Drug Delivery Rev., 1:199-233 (1987); Langer, “New Methods of Drug Delivery”, Science, 249:1527-33 (1990); Chien et al., Novel Drug Delivery Systems (1982). Such delivery systems offer the potential of enhanced therapeutic efficacy and reduced overall toxicity.

If an implant is intended for use as a drug delivery or other controlled-release system, using a biodegradable polymeric carrier is one effective means to deliver the therapeutic agent locally and in a controlled fashion, see Langer et al., “Chemical and Physical Structures of Polymers as Carriers for Controlled Release of Bioactive Agents”, J. Macro. Science, Rev. Macro. Chem. Phys., C23(1), 61-126 (1983). As a result, less total drug is required, and toxic side effects can be minimized. Examples of classes of synthetic polymers that have been studied as possible solid biodegradable materials include polyesters (Pitt et al., “Biodegradable Drug Delivery Systems Based on Aliphatic Polyesters: Applications to Contraceptives and Narcotic Antagonists”, Controlled Release of Bioactive Materials, 19-44 (Richard Baker ed., 1980); poly(amino acids) and pseudo-poly(amino acids) (Pulapura et al. “Trends in the Development of Bioresorbable Polymers for Medical Applications”, J. Biomaterials Appl., 6:1, 216-50 (1992); polyurethanes (Bruin et al., “Biodegradable Lysine Diisocyanate-based Poly(Glycolide-co-.epsilon. Caprolactone)-Urethane Network in Artificial Skin”, Biomaterials, 11:4, 291-95 (1990); polyorthoesters (Heller et al., “Release of Norethindrone from Poly(Ortho Esters)”, Polymer Engineering Sci., 21:11, 727-31 (1981); and polyanhydrides (Leong et al., “Polyanhydrides for Controlled Release of Bioactive Agents”, Biomaterials 7:5, 364-71 (1986).

Thus, for example, an Epac, PLCε, or PLD inhibitor composition can be incorporated into a biocompatible polymeric composition and formed into the desired shape outside the body. This solid implant is then typically inserted into the body of the subject through an incision. Alternatively, small discrete particles composed of these polymeric compositions can be injected into the body, e.g., using a syringe. In an exemplary embodiment, an inhibitor composition can be encapsulated in microspheres of poly(D,L-lactide)polymer suspended in a diluent of water, mannitol, carboxymethyl-cellulose, and polysorbate 80. The polylactide polymer is gradually metabolized to carbon dioxide and water, releasing the inhibitor into the system.

In yet another approach, depot formulations can be injected via syringe as a liquid polymeric composition. Liquid polymeric compositions useful for biodegradable controlled release drug delivery systems are described, e.g., in U.S. Pat. Nos. 4,938,763; 5,702,716; 5,744,153; 5,990,194; and 5,324,519. After injection in a liquid state or, alternatively, as a solution, the composition coagulates into a solid.

One type of polymeric composition suitable for this application includes a nonreactive thermoplastic polymer or copolymer dissolved in a body fluid-dispersible solvent. This polymeric solution is placed into the body where the polymer congeals or precipitates and solidifies upon the dissipation or diffusion of the solvent into the surrounding body tissues. See, e.g., Dunn et al., U.S. Pat. Nos. 5,278,201; 5,278,202; and 5,340,849 (disclosing a thermoplastic drug delivery system in which a solid, linear-chain, biodegradable polymer or copolymer is dissolved in a solvent to form a liquid solution).

An Epac, PLCε, or PLD inhibitor composition can also be adsorbed onto a membrane, such as a silastic membrane, which can be implanted, as described in International Publication No. WO 91/04014.

D. Dose

The dose of inhibitor is sufficient to inhibit the target (i.e., Epac, PLCε, or PLD), preferably without significant toxicity. In particular in vivo embodiments, the amount of the inhibitor is sufficient to reduce pain in a subject. In variations of this embodiment in which an analgesic or other agent (such as, e.g., an anticonvulsant, antidepressant, and/or neuroleptic) is co-administered with the inhibitor, the amount of the analgesic or other agent is sufficient produce a beneficial effect in the subject (i.e., an analgesic effect, in the case of an analgesic). For in vivo applications, the dose of inhibitor and any other, optional, agent depends, for example, upon the therapeutic objectives, the route of administration, and the condition of the subject. Accordingly, it is necessary for the clinician to titer the dosage and modify the route of administration as required to obtain the optimal therapeutic effect. Generally, the clinician begins with a low dose and increases the dosage until the desired therapeutic effect is achieved. Starting doses for a given inhibitor can be extrapolated from in vitro data.

Methods of Screening for Agents that Reduce Pain

The role of Epac, PLCε, and PLD in mediating pain makes these molecules attractive targets for agents that can reduce pain. Accordingly, the invention provides prescreening and screening methods aimed at identifying such agents. Test agents can be prescreened, for example, based on binding to Epac, PLCε, and/or PLD or on binding to a polynucleotide encoding any of these polypeptides. Screening methods of the invention can be carried out by: contacting a test agent with Epac, PLCε, and/or PLD; determining whether the test agent inhibits Epac, PLCε, and/or PLD, respectively; and if so, selecting the test agent as a potential analgesic. For example, test agents can be screened for effects on the levels of Epac, PLCε, and/or PLD or polynucleotides encoding them (e.g., Epac, PLCε, and/or PLD mRNA) or for effects on Epac, PLCε, and/or PLD function.

The prescreening/screening methods of the invention are generally, although not necessarily, carried out in vitro. Accordingly, screening assays are generally carried out, for example, using purified or partially purified components in cell lysates or fractions thereof, in cultured cells, or in a biological sample, such as a tissue or a fraction thereof.

A. Prescreening Based on Binding to Epac, PLCε, and/or PLD

The invention provides a prescreening method based on assaying test agents for specific binding to Epac, PLCε, and/or PLD. Agents that specifically bind to Epac, PLCε, and/or PLD have the potential to modulate function and thereby modulate pain.

In one embodiment, therefore, a prescreening method of the invention entails contacting a test agent with Epac, PLCε, and/or PLD. Specific binding of the test agent to the contacted polypeptide is then determined. If specific binding is detected, the test agent is selected as a potential analgesic.

Such prescreening is generally most conveniently accomplished with a simple in vitro binding assay. Means of assaying for specific binding of a test agent to a polypeptide are well known to those of skill in the art. In preferred binding assays, the polypeptide is immobilized and exposed to a test agent (which can be labeled), or alternatively, the test agent(s) are immobilized and exposed to the polypeptide (which can be labeled). The immobilized species is then washed to remove any unbound material and the bound material is detected. To prescreen large numbers of test agents, high throughput assays are generally preferred. Various assay formats are discussed in greater detail below.

B. Prescreening Based on Binding to Polynucleotides Encoding Epac, PLCε, and/or PLD

The invention also provides a prescreening method based on screening test agents for specific binding to a polynucleotide encoding Epac, PLCε, and/or PLD. Agents that specifically bind to such polynucleotides have the potential to modulate the expression of the encoded polypeptide, and thereby modulate pain.

In one embodiment, therefore, a prescreening method of the invention entails contacting a test agent with a polynucleotide encoding Epac, PLCε, and/or PLD. Specific binding of the test agent to the polynucleotide is then determined. If specific binding is detected, the test agent is selected as a potential analgesic.

Such prescreening is generally most conveniently accomplished with a simple in vitro binding assay, which are well known to those of skill in the art. In preferred binding assays, the polynucleotide is immobilized and exposed to a test agent (which can be labeled), or alternatively, the test agent(s) are immobilized and exposed to the polynucleotide (which can be labeled). The immobilized species is then washed to remove any unbound material and the bound material is detected. To prescreen large numbers of test agents, high throughput assays are generally preferred. Various assay formats are discussed in greater detail below.

C. Screening Based on Levels of Epac, PLCε, and/or PLD or on Levels of Epac, PLCε, and/or PLD Polynucleotides

Test agents, including, for example, those identified in a prescreening assay of the invention can also be screened to determine whether the test agent affects the level(s) of Epac, PLCε, and/or PLD or the level(s) of polynucleotides encoding any of these polypeptides (e.g., Epac, PLCε, and/or PLD mRNA). Agents that reduce these levels can potentially reduce pain.

Accordingly, the invention provides a method of screening for an agent that reduces pain in which a test agent is contacted with a cell that expresses Epac, PLCε, and/or PLD in the absence of test agent. Preferably, the method is carried out using an in vitro assay. In such assays, the test agent can be contacted with a cell in culture or present in a tissue. Alternatively, the test agent can be contacted with a cell lysate or fraction thereof. The level of Epac, PLCε, and/or PLD polypeptides or polynucleotides (e.g., mRNA) is determined in the presence and absence (or presence of a lower amount) of test agent to identify any test agents that alter the level. If the level is reduced, the test agent is selected as a potential analgesic.

Cells or tissues useful in this screening method include those from any of the species described above in connection with the method of reducing pain. Cells that naturally express Epac, PLCε, and/or PLD are typically, although not necessarily, employed in this screening method. Examples include, but are not limited to, cultured dorsal root ganglia cells, as described in Example 1. Examples of cells useful in screening for agents that act via Epac include microglia, pancreatic β-cells, retinal neurons, stem cells, and leukocytes. Screening for agents that act via PLCε can be carried out, for example, in N1E115 neuroblastoma cells, HEK293 cells, and corneal epithelial cells. Examples of cells useful in screening for agents that act via PLD include PC12 cells, neural stem cells, myocytes, C6 glioma cells, Ewing sarcoma cells, renal epithelial cells, and cerebellar ganule cells. Alternatively, cells that have been engineered to express Epac, PLCε, and/or PLD can be used in the method. 1. Sample

As noted above, screening assays are generally carried out in vitro, for example, in cultured cells, in a biological sample (e.g., brain), or fractions thereof. For ease of description, cell cultures, biological samples, and fractions are referred to as “samples” below. The sample is generally derived from an animal (e.g., any of the research animals mentioned above), preferably from a mammal, and more preferably from a human.

The sample may be pretreated as necessary by dilution in an appropriate buffer solution or concentrated, if desired. Any of a number of standard aqueous buffer solutions, employing one or more of a variety of buffers, such as phosphate, Tris, or the like, at physiological pH can be used.

2. Polypeptide-Based Assays

Epac, PLCε, and/or PLD can be detected and quantified by any of a number of methods well known to those of skill in the art. Examples of analytic biochemical methods suitable for detecting such polypeptides include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

In one embodiment, Epac, PLCε, and/or PLD are detected/quantified using a binding assay. Briefly, a sample from a tissue expressing the polypeptide of interest is incubated with a suitable binding partner (such as, e.g., an antibody) under conditions designed to provide a saturating concentration of the binding partner over the incubation period. After treatment with the binding partner, the sample is assayed for binding. Any binding partner that binds to the polypeptide of interest can be employed in the assay, although, if the polypeptide is one of a number of isoforms, binding partners specific for the particular isoform being assayed are preferred. In addition to antibodies, any Epac, PLCε, and/or PLD inhibitor that binds directly to Epac, PLCε, and/or PLD can, for example, be labeled and used in this assay. Exemplary binding partners for Epac and PLD are described in Example 1, namely a polyclonal rabbit anti-Epac1 serum from Santa Cruz Biotechnology and the PLD inhibitor 1-butanol.

In another embodiment, Epac, PLCε, and/or PLD are detected/quantified in an electrophoretic polypeptide separation (e.g. a 1- or 2-dimensional electrophoresis). Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence of Epac, PLCε, and/or PLD in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the support with antibodies that specifically bind the target polypeptide(s). Antibodies that specifically bind to the target polypeptide(s) may be directly labeled or alternatively may be detected subsequently using labeled antibodies (e.g., labeled sheep anti-mouse antibodies) that specifically bind to a domain of the primary antibody.

In a preferred embodiment, Epac, PLCε, and/or PLD are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Conventional immunoassays often utilize a “capture agent” to specifically bind to and often immobilize the analyte (in this case Epac, PLCε, or PLD). In preferred embodiments, the capture agent is an antibody.

Immunoassays also typically utilize a labeling agent to specifically bind to and label the binding complex formed by the capture agent and the target polypeptide. The labeling agent may itself be one of the moieties making up the antibody/target polypeptide complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/target polypeptide complex. Other polypeptides capable of specifically binding immunoglobulin constant regions, such as polypeptide A or polypeptide G may also be used as the labeling agent. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured target polypeptide is directly measured. In competitive assays, the amount of target polypeptide in the sample is measured indirectly by measuring the amount of an added (exogenous) polypeptide displaced (or competed away) from a capture agent by the target polypeptide present in the sample. In one competitive assay, a known amount of, in this case, labeled Epac, PLCε, or PLD is added to the sample, and the sample is then contacted with a capture agent. The amount of labeled Epac, PLCε, or PLD bound to the antibody is inversely proportional to the concentration of polypeptide present in the sample.

Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads™), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., ³H, ¹²⁵I, ³⁵ s, ¹⁴C, or ³²P), enzymes (e.g., horseradish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

In particular embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen, or DNA fragment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Antibodies useful in these immunoassays include polyclonal and monoclonal antibodies.

3. Polynucleotide-Based Assays

Changes in Epac, PLCε, and/or PLD expression level can be detected by measuring changes in levels of mRNA and/or a polynucleotide derived from the mRNA (e.g., reverse-transcribed cDNA, etc.).

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

i. Amplification-Based Assays

In one embodiment, amplification-based assays can be used to detect, and optionally quantify, a polynucleotide encoding Epac, PLCε, and/or PLD. In exemplary amplification-based assays, Epac, PLCε, and/or PLD mRNA in the sample acts as a template in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of Epac, PLCε, and/or PLD mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

ii. Hybridization-Based Assays

Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of Epac, PLCε, and/or PLD mRNA can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of Epac, PLCε, and/or PLD mRNA, and standard methods (such as, e.g., densitometry where the nucleic acid probe is radioactively labeled) can be used to quantify the level of Epac, PLCε, and/or PLD mRNA).

A variety of additional nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to a solid support and a labeled “signal” nucleic acid in solution. The sample provides the target polynucleotide. The capture nucleic acid and signal nucleic acid each hybridize with the target polynucleotide to form a “sandwich” hybridization complex.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low-density” arrays can simply be produced by spotting (e.g., by hand using a pipette) different nucleic acids at different locations on a solid support (e.g., a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

In a preferred embodiment, the arrays used in this invention contain “probe” nucleic acids. These probes are then hybridized respectively with their “target” nucleotide sequence(s) present in polynucleotides derived from a biological sample. Alternatively, the format can be reversed, such that polynucleotides from different samples are arrayed and this array is then probed with one or more probes, which can be differentially labeled.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, and/or enhance signal detection. If covalent bonding between a compound and the surface is desired, the surface will usually be polyfunctional or be capable of being polyfunctionalized. Functional groups that may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

Arrays can be made up of target elements of various sizes, ranging from about 1 mm diameter down to about 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

Hybridization assays according to the invention can also be carried out using a MicroElectroMechanical System (MEMS), such as the Protiveris' multicantilever array.

iii. Polynucleotide Detection

Epac, PLCε, and/or PLD polynucleotides can be detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods described in the art are the nucleic acid sequence based amplification (NASBAO, Cangene, Mississauga, Ontario) and Q Beta Replicase systems.

In a preferred embodiment, suitable for use in amplification-based assays of the invention, a primer contains two fluorescent dyes, a “reporter dye” and a “quencher dye.” When intact, the primer produces very low levels of fluorescence because of the quencher dye effect. When the primer is cleaved or degraded (e.g., by exonuclease activity of a polymerase, see below), the reporter dye fluoresces and is detected by a suitable fluorescent detection system. Amplification by a number of techniques (PCR, RT-PCR, RCA, or other amplification method) is performed using a suitable DNA polymerase with both polymerase and exonuclease activity (e.g., Taq DNA polymerase). This polymerase synthesizes new DNA strands and, in the process, degrades the labeled primer, resulting in an increase in fluorescence. Commercially available fluorescent detection systems of this type include the ABI Prism® Systems 7000, 7700, or 7900 (TaqMan®) from Applied Biosystems or the LightCycler® System from Roche.

D. Screening Based on Epac, PLCε, or PLD Action

The invention also provides a screening method based on determining the effect, if any, of a test agent on the level of Epac, PLCε, and/or PLD action. Epac, PLCε, and/or PLD action can be assayed my measuring any activity of, or response mediated by Epac, PLCε, and/or PLD. Agents that reduce Epac, PLCε, and/or PLD action can potentially reduce pain.

Accordingly, the invention provides a method of screening for an agent that can reduce pain in which a test agent is contacted with a cell that expresses Epac, PLCε, and/or PLD, or a fraction thereof, in the absence of test agent. Preferably, the method is carried out using an in vitro assay. When the test agent can be contacted with a cell, the cell can be in culture or present in a tissue. The level of Epac, PLCε, and/or PLD action is determined in the presence and absence (or presence of a lower amount) of test agent to identify any test agents that alter the level. If the level of Epac, PLCε, and/or PLD action is reduced, the test agent is selected as a potential analgesic.

Cells or tissues useful for screening based on Epac, PLCε, and/or PLD action include any of those described above in connection with screening based on levels of Epac, PLCε, and/or PLD or on levels the polynucleotides encoding any of these polypeptides.

Epac action can be measured using any assay for an Epac activity or Epac-mediated response. Examples of suitable assays include the measurement of: Epac-cAMP binding, Epac-mediated activation of the GTPase Rap1, Epac-mediated activation of a MAP kinase.

PLCε action can be measured using any assay for a PLCε activity or PLCε-mediated response. For example, the screening method can assay the effect of a test agent on PLCε-mediated hydrolysis of phosphotidylinositol to inositol triphosphate and diacylglycerol (DAG).

PLD action can be measured using any assay for a PLD activity or PLD-mediated response. For example, the screening method can assay the effect of a test agent on PLD-mediated hydrolysis of a phospholipid substrate (e.g., phosphatidylcholine, phosphatidylethanolamine, phosphatidylinositol, phosphatidylserine, lyso phosphatidylcholine, sphingomyelin, phosphotidylglycerol, or N-acyl phosphatidylethanolamine) to phosphatidic acid. See, e.g., Petersen, G. et al. (2000) J Lipid Res. 41:1532.

E. Test Agent Databases

In a preferred embodiment, generally involving the screening of a large number of test agents, the screening method includes the recordation of any test agent selected in any of the above-described prescreening or screening methods in a database of candidate analgesics.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

F. Test Agents Identified by Screening

When a test agent is found to alter the level of Epac, PLCε, and/or PLD; polynucleotides encoding Epac, PLCε, and/or PLD; or Epac, PLCε, and/or PLD action, a preferred screening method of the invention further includes combining the test agent with a carrier, preferably a pharmaceutically acceptable carrier, such as are described above. Generally, the concentration of test agent is sufficient to alter the level of Epac, PLCε, and/or PLD or their respective polynucleotides or actions when the composition is contacted with a cell. This concentration will vary, depending on the particular test agent and specific application for which the composition is intended. As one skilled in the art appreciates, the considerations affecting the formulation of a test agent with a carrier are generally the same as described above with respect to methods of reducing pain.

In a preferred embodiment, the test agent is administered to an animal to measure the ability of the selected test agent to reduce pain in a subject, as described in greater detail below.

G. Screening Based on Reduction of Pain in vivo

The invention also provides an in vivo method of screening for an agent that that can reduce pain in a subject. The method entails selecting an Epac inhibitor, a PLCε inhibitor, or a PLD inhibitor as a test agent, and measuring the ability of the selected test agent to reduce pain in a subject. Any agent that inhibits Epac, PLCε, and/or PLD and that can be administered to a subject can be employed in the method. Accordingly, test agents selected through any of the prescreening or screening methods of the invention can be tested in vivo. Alternatively, known inhibitors of Epac, PLCε, and/or PLD can be employed.

Test agents can be formulated for administration to a subject as described above for Epac, PLCε, and/or PLD inhibitors.

The subject of the method can be any individual that has Epac, PLCε, and/or PLD and in which symptoms of pain can be measured. Examples of suitable subjects include research animals, such as mice, rats, guinea pigs, rabbits, cats, dogs, as well as monkeys and other primates, and humans. In preferred embodiments, an animal model established for studying a response to pain is employed. For instance, the animal model can be one that tests the nociceptive flexion reflex, as described in Example 1.

Generally, the test agent is administered to the subject before application of a painful stimulus, and the subject is tested or observed to determine whether the test agent reduces a particular response to the stimulus. I.e., the response is measured and compared with that observed in the absence of test agent and/or in the presence of a lower amount of test agent. Test agents can be administered by any suitable route, as described above for Epac, PLCε, and/or PLD inhibitors. Generally, the concentration of test agent is sufficient to alter the level of Epac, PLCε, and/or PLD polypeptides, polynucleotides, or action in vivo.

Kit

The invention also provides kits useful in practicing the methods of the invention. In one embodiment, a kit of the invention includes a Epac, PLCε, and/or PLD inhibitor in a suitable container. In a variation of this embodiment, the inhibitor is formulated in a pharmaceutically acceptable carrier. The kit preferably includes instructions for administering the inhibitor to a subject to reduce pain.

Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

The following examples are offered to illustrate, but not to limit, the claimed invention.

### Example 1

### Epac Mediates cAMP to PKC Signaling in Inflammatory Pain: an IB4(+)-Neuron Specific Mechanism

Abstract

The epsilon isoform of PKC (PKCε) has emerged as a critical second messenger in sensitization toward mechanical stimulation in models of neuropathic (diabetes, alcoholism, cancer-therapy), as well as acute and chronic inflammatory pain. Signaling pathways leading to activation of PKCε remained unknown. Recent results indicate signaling from cAMP to PKC. A mechanism connecting cAMP and PKC, two ubiquitous, commonly considered separate pathways, remained elusive. The present work demonstrated, in cultured dorsal root ganglion (DRG) neurons, that signaling from cAMP to PKCε is not mediated by protein kinase A (PKA) but by the recently identified cAMP-activated guanine exchange factor, Epac. Epac, in turn, was upstream of phospholipase C (PLC) and phospholipase D (PLD), both of which were necessary for translocation and activation of PKCε. This signaling pathway was specific to isolectin B4-positive (IB4(+)) nociceptors. Also, in a behavioral model, cAMP produced mechanical hyperalgesia (tenderness) through Epac, PLC/PLD and PKCε. By delineating this signaling pathway this work provides a mechanism for cAMP to PKC signaling, gives proof of principle that the mitogen-activated protein kinase (MAPK) pathway activating protein Epac also stimulates PKC, describe the first physiological function unique for the EB4(+) subpopulation of sensory neurons, and find proof of principle that G-protein coupled receptors can activate PKC not only through the G-proteins α_{q} and βγ but also through αₛ.

Methods

Antibodies

Antibodies and lectins used in this study were: PKCε-specific monoclonal mouse antibody from BD Transduction Laboratories; β₂-adrenergic receptor (β₂-AR)-specific polyclonal rabbit antiserum, polyclonal rabbit anti-Epac1 serum from Santa Cruz Biotechnology; monoclonal mouse NeuN-specific antibody from Chemicon; IB4-AlexaFluor-568 from Molecular Probes; donkey rabbit-specific FITC-conjugated, donkey mouse-specific Rhodamine-conjugated secondary antibody from Jackson Immunoresearch; and PKCε-specific rabbit serum (SN134) provided by Dr. Robert Messing, UCSF.

Drugs

1-butanol, alprenolol, D-609, epinephrine, forskolin, (−)-isoproterenol, and U-73343 were purchased from Sigma; 2-butanol from Fluka; bisindolylmaleimide I, cholera toxin, εV1-2, U-73122, 4-cyano-3-methylisoquinoline, 8-CPT-2′-O-Me-cAMP, 8-CPT from Calbiochem; Nembutal from Abbott Laboratories; ICI 118,551 from Tocris Cookson, and PKA catalytic subunit from New England BioLabs.

Chemicals

Chemicals used in this study were: trypsin from Worthington Biochemical Corporation; collagenase from Boehringer Mannheim; NeurobasalA (w/o phenol red), B27 from Invitrogen; glutamine, MEM-medium, Hanks-medium, penicillin/streptavidin solution from UCSF Cell Culture Facility; BSA, DMSO, glutamate, para-formaldehyde, TritonX-100 from Sigma; and normal goat serum, Vectashield from Vector Laboratories.

Animals

Behavioral experiments were performed on male Sprague-Dawley rats (200-300 g, Charles River, Hollister, Calif., USA). Animals were housed in a controlled environment in the Animal Care Facility of the University of California, San Francisco, under a 12 h light/dark cycle. Food and water were available ad libitum. Care and use of animals conformed to NIH guidelines. The UCSF Committee on Animal Research approved experimental protocols. All efforts were made to minimize the number of animals used.

DRG-Cultures

Cultures of dissociated dorsal root ganglia were prepared from male Sprague-Dawley rats (200-300 g, Charles River, Hollister, Calif., USA), adapting a previously described protocol (Khasar et al., 1999b). Rats were anesthetized with an overdose of Nembutal (50 mg per animal, s.c.). L1-L6 DRGs were removed, desheathed, pooled, incubated with collagenase (final concentration (f.c.) 0.125%, 1 h, 37° C.) followed by a trypsin digest (f.c. 0.25%, 7 min, 37° C.). Cells were separated by trituration with a fire polished Pasteur pipette. Axon stumps and dead cells were removed by straining (40 μm mesh), followed by centrifugation (3 min, 500 g). Cells were resuspended in 12 ml Neurobasal A/B27-media and plated 0.5 ml (=0.5 DRG equivalents) per culture onto polyornithine/laminin precoated glass cover slips (12 mm diameter) and incubated over night in 24-well plates at 37° C. in 5% CO₂. Variability between batches of NeurobasalA media and its B27 supplement resulting in varying translocation of PKCε after stimulation was counteracted by the addition of up to 1 μM ethanol (according to technical support of Invitrogen, less than 2.5% of the ethanol concentration already in the medium).

Stimulation

After a 15-20 h incubation, to allow cells to adhere to coverslips, the cells were stimulated. To ensure homogeneous dispersion of the stimulants, 250 μl of the 500 μl medium was removed, mixed thoroughly with the respective activator/inhibitor and added back to the same culture. Inhibitors were added at a concentration of 10-times IC₅₀-values, 15 min before stimulation. Activators were added for the indicated time in concentrations based on literature reports. For all agonists time courses were established (some data not shown). Negative controls (unstimulated cells) were treated alike only without the addition of any pharmaceutical reagent. While the physiological stimulus epinephrine was used for the in vivo experiments, the β-AR agonist isoproterenol was used in vitro for identification of the β-AR subtype. After treatment the cells were washed once with phosphate buffered saline (PBS) and fixed with para-formaldehyde (PFA) (4%, 10 min, room temperature (RT)).

Immunocytochemistry

PFA-fixed cells were permeabilized with 0.1% Triton X-100 (10 min, RT), followed by three washes with 0.1% bovine serum albumin (BSA)/PBS (5 min, RT). After blockage of unspecific binding sites (5% BSA/10% normal goat serum/PBS, 1 h, RT) the cultures were probed with the respective primary antibody in 1% BSA/PBS (over night, 4° C.), washed three times (1% BSA/PBS, 5 min, RT) and incubated with the secondary fluorophore-coupled antiserum (final concentration (f.c.) 1:200, 1 h, RT). After three final washes (PBS, 5 min, RT), the cultures were mounted with Vectashield onto microscope slides and sealed with nail polish.

Evaluation of PKCε Translocation

Cells were evaluated with a Nikon Microphot FXA microscope, using a 50× oil objective. 50 randomly selected cells per culture were evaluated. Data are plotted as mean percentage of translocating cells per evaluated culture±standard error of means (SEM) based on the number of evaluated cultures. All counting was done in blind fashion by the same observer. All treatments have been repeated with DRG-neurons from different rats, on at least 2 separate days. Confocal images were taken with a 100× oil immersion objective using a Zeiss Axiovert 100 microscope (Carl Zeiss Inc.) attached to a MRC 1000 confocal microscope (Bio-Rad). Epifluorescent images were taken with a 63× oil immersion objective using a Zeiss Axiovert 100 microscope.

Testing of Mechanical Nociceptive Threshold

The nociceptive flexion reflex was quantified using the Randall-Selitto paw pressure device (Analgesymeter®, Stoetling), which applies a linearly increasing mechanical force to the dorsum of the rat's hind paw. The nociceptive mechanical threshold was defined as the force in grams at which the rat withdrew its paw. The protocols for this procedure have been previously described (Taiwo and Levine, 1989; Dina et al., 2003). Baseline paw-withdrawal threshold was defined as the mean of six readings before test agents were injected. Each paw was treated as an independent measure and each experiment was performed on a separate group of rats. Each group of rats was treated with only one agonist and/or antagonist injected peripherally by the intradermal route. Measurement of nociceptive threshold was taken 30 min after the administration of the hyperalgesic mediator. All behavioral testing was done between 10.00 and 16.00 h. The blocking agents were injected as described previously (Khasar et al., 1995; Khasar et al., 1999a). Because it is less membrane permeable, injections of the PKCε inhibitor (εV1-2) (Johnson et al., 1996) and the PKA catalytic subunit (Slice and Taylor, 1989) was always preceded by administration of 2.5 μL of distilled water in the same syringe, separated by a small air bubble, to produce hypo-osmotic shock, thereby enhancing cell membrane permeability to the drug (Khasar et al., 1995; Khasar et al., 1999a). The onset of mechanical hyperalgesia is statistically significant already after 2 minutes (Khasar et al., 1999a) mirroring the cellular results, the mechanical threshold was tested 30 minutes after injection of the respective stimulus, a time point of plateauing maximal response.

Statistical Analysis

All statistical comparisons were made with one way ANOVAs followed by Dunnet's multiple comparison post hoc test, the p values of which are given.

Results

β₂-Adrenergic Receptor Activation Translocates PKCε in DRG Neurons

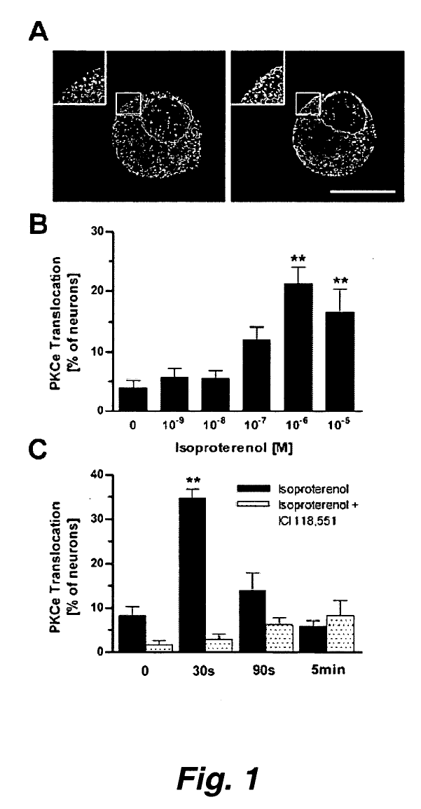

To investigate the second messenger-signaling pathway upstream of PKCε translocation of PKCε was evaluated in dissociated dorsal root ganglion neurons, a central step in activation of PKCs (Dorn and Mochly-Rosen, 2002) and an established surrogate measurement of PKC activation (Cesare et al., 1999). As observed by stimulation with bradykinin and phorbol ester (Cesare et al., 1999) also the β-adrenergic receptor (β-AR) agonist isoproterenol induced translocation of PKCε to the plasma membrane of DRG neurons (FIG. 1A). Because of the intense cytoplasmic PKCε signal potential translocation to other intracellular targets was not evaluated.

A dose response curve established that 1 μM isoproterenol produced translocation in a maximal number of cells (cultures evaluated n=8, 21.3%±2.8% PKCε translocating cells, FIG. 1B). Translocation of PKCε was transient, peaking at about 30 s, decaying by 90 s and returning to baseline after 5 min (FIG. 1C). Induction of translocation by isoproterenol was mediated by β₂-AR as it was inhibited by the β₂-AR specific antagonist ICI 118,551 (n=8, 3.0%±1.1% PKCε translocating cells, FIG. 1C). ICI 118,551 is described as an inverse agonist not only blocking receptor activation but also reducing its baseline activity (Bond et al., 1995), reflected by the decrease in baseline PKCε translocation.

PKCε Translocation is Mediated by αₛ and AC but not by PKA

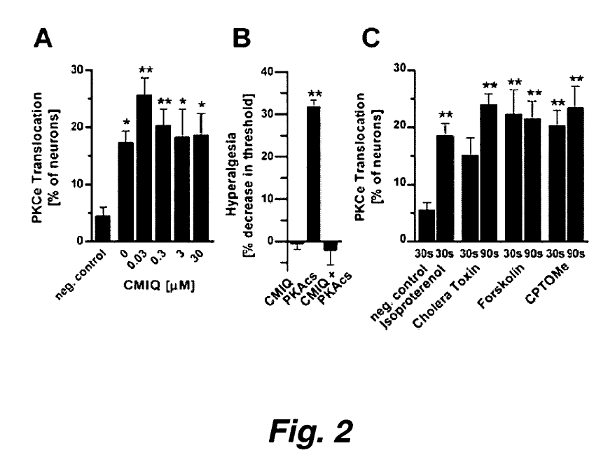

To establish that the β₂-AR signals to PKC through cAMP, the well described activator of αₛ, cholera toxin, was applied to the cell-cultures, classically leading to a rise in intracellular cAMP. Translocation of PKCε to the plasma membrane was observed starting 30 s after treatment and peaking by 90 s (n=6, 15.2±3.0% and 24.0±1.9%, respectively, of neurons showing PKCε translocation (FIG. 2B)).

Since αₛ activates AC (Neves et al., 2002), the well-established activator of AC, forskolin, was used to test for its involvement in the signaling cascade leading to PKCε translocation. With a maximal response time of 30 s, forskolin also induced the translocation of PKCε to the plasma membrane (n=6, 22.3±4.2% PKCε translocating cells (FIG. 2B)).

Since cAMP activates PKA, the involvement of PKA in isoproterenol-induced PKCε activation was examined. As the commonly used PKA inhibitor H89 inhibits also ligand binding to the β₂-AR (Penn et al., 1999), and inactive cAMP analogs such as Rp-cAMP risk also inhibiting other cAMP effectors, the PKA specific membrane permeable inhibitor 4-cyano-3-methylisoquinoline (CMIQ) was used. This inhibitor blocks the ATP binding site of PKA (Lu et al., 1996). 15 minute preincubation with CMIQ completely abrogated hyperalgesia in vivo induced by injection of the catalytic subunit of PKA (Slice and Taylor, 1989) (FIG. 2B). Therefore the DRG cultures were preincubated with CMIQ for 15 minutes with concentrations up to 1000-fold greater than its IC₅₀ value, before stimulation with isoproterenol. Intriguingly, in the presence of CMIQ, PKCε still translocated to the plasma membrane, as in control conditions (n=6, FIG. 2A). Thus, while αₛ and AC are involved in the signal transduction from the β₂-AR to PKCε, PKA is not.

Epac Mediates PKCε Translocation

Since cAMP was not signaling through PKA to induce PKCε activity, the possibility that cAMP activation of Epac leads to translocation of PKCε in dissociated DRG neurons was examined. An Epac specific activator, the cAMP analog 8-CPT-2′-O-Me-cAMP (CPTOMe), has been developed for the differentiation of PKA and Epac mediated effects (Enserink et al., 2002; Rehmann et al., 2003). Stimulating neurons with this compound led to robust translocation of PKCε to the plasma membrane as seen with the activators of β₂-AR, αₛ, and AC, corroborating the observation that inhibition of PKA does not change the extent of PKCε translocation (FIG. 2B), and, for the first time, placing Epac upstream of PKC.

As CPTOMe induced a maximal number of cells with translocated PKCε at 90 s (n=6, 20.3±2.6% versus 23.5±3.7% translocating neurons after CPTOMe-stimulation for 30 s and 90 s, respectively), this time point was employed in the subsequent investigation of downstream events of CPTOMe stimulation of Epac.

PKCε Translocation is PLC and PLD Dependent

DAG, a product of members of the PLC-family, can activate novel PKCs, such as PKCε. Accordingly, the involvement of phosphatidylcholate (PC) and phosphatidylinositol (PI) hydrolyzing PLCs, as well as the involvement of phospholipase D, in Epac-induced PKCε translocation were examined.

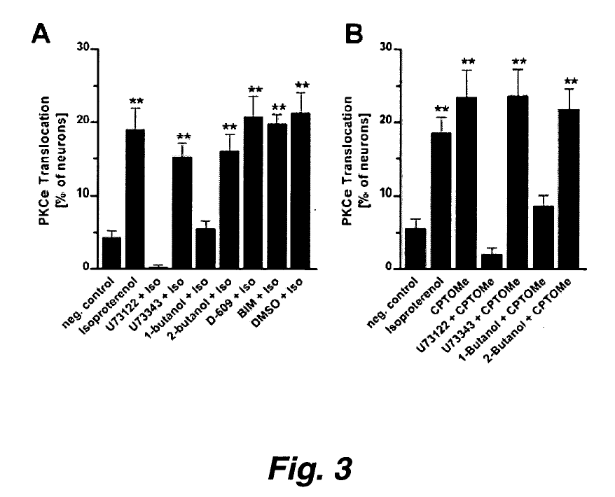

To check for the involvement of PI—PLC, of which PLCε is a member (Schmidt et al., 2001), the cultures were preincubated for 15 min with the PI—PLC inhibitor U73122 before stimulating with isoproterenol for 30 s. The translocation of PKCε was completely blocked by this inhibitor. In contrast, the inactive analogue of U73122, U73343, produced only a slight reduction in translocation (n=8, 0.3±0.3%, and 15.3±1.9% PKCε translocating cells in U73122 versus U73343, pretreated cultures;19.0±3.0% PKCε translocating cells in isoproterenol controls (FIG. 3A)).

While the PI—PLC inhibitor U73122 and its negative control U73343 were solubilized in DMSO, by itself DMSO does not show any inhibitory influence over a wide range of concentrations as exemplified here by the highest concentration used (dilution 1:200, n=8, 21.3±2.9% PKCε translocating cells, FIG. 3A).

In addition, DAG can be produced indirectly by PLD, which produces phosphatidic acid (PA) that is metabolized to DAG (Rizzo and Romero, 2002). Also, PLD produced PA has been shown, in RBL-2H3 cells, to act on PKCε directly (Jose Lopez-Andreo et al., 2003). Therefore, cultures were pretreated with the competitive PLD inhibitor 1-butanol or its not interfering control, 2-butanol, for 15 min, and then stimulated with 1 μM isoproterenol for 30 s. The use of 1-butanol but not 2-butanol results in a decrease in the number of PKCε translocating cells, to baseline levels (n=8, 5.5±1.1% PKCε translocating cells in 1-butanol versus 16.0±2.4% in 2-butanol pretreated cultures, FIG. 3A).

The compound D-609 has been shown to block PC hydrolyzing PLCs (PC—PLC) while leaving the activity of PI—PLC and other PLCs unchanged (Schutze et al., 1992). Preincubation of DRG neuron cultures with D-609 for 15 min before stimulating with isoproterenol, for 30 s, produced no change in isoproterenol-induced translocation of PKCε (n=8, 20.8±2.9% (D-609 pretreated) PKCε translocating cells in contrast to 19.0±3.0% in isoproterenol controls, FIG. 3A), suggesting that PC—PLCs are not involved in the activation of PKCε. Thus, both PI—PLC and PLD, but not PC—PLC, are necessary for the isoproterenol-induced translocation of PKCε.

To exclude that the activity of PLC and PLD leads to the activation of a PKC subtype different than PKCε, which then in turn contributes to the activation of PKCε, the effect PKC inhibitor bisindolylmaleimide I (BIM) was studied. BIM inhibits PKC activity by blocking the ATP-binding site but which does not inhibit translocation of PKCs in the course of activation (Toullec et al., 1991). Again, the cultures were pretreated for 15 min with the inhibitor before stimulation with isoproterenol. There was no reduction in cells showing PKCε translocation (n=8, PKCε translocation in 19.8±1.3% in BIM treated, versus 19.0±3.0% in untreated isoproterenol stimulated cells, FIG. 3A). Therefore, PI—PLC and PLD are acting not via a different PKC subtype but on PKCε directly.

PLC and PLD are Downstream of Epac

To investigate if PI—PLC and PLD are downstream of the αₛ/AC/cAMP/Epac signaling pathway delineated with the earlier experiments and not downstream of a possible parallel signaling cascade (e.g. transactivation of a receptor tyrosine kinase leading to lipase activation (Luttrell et al., 1997; Lee and Chao, 2001), the influence of U73122 and U73343 or 1- and 2-butanol on PKCε translocation was evaluated after direct activation of Epac with CPTOMe. Again, cultures were pretreated with the respective inhibitor or its inactive control compound for 15 min before adding the Epac activator, CPTOMe, and stimulating for 90 s. As shown in FIG. 3B, the activity of both phospholipases is also necessary for the translocation of PKCε to occur in response to direct activation of Epac (n=6, PKCε translocation in 2.0±0.9% of U73122, 23.7±3.6% of U73343, 8.7±1.5% of 1-butanol, 21.8±2.7% of 2-butanol pretreated and CPTOMe stimulated cells, and 23.5±3.7% in CPTOMe stimulated cells, FIG. 3B).

β₂-Adrenergic Receptor-Induced Translocation of PKCε Occurs in IB4+ Neurons

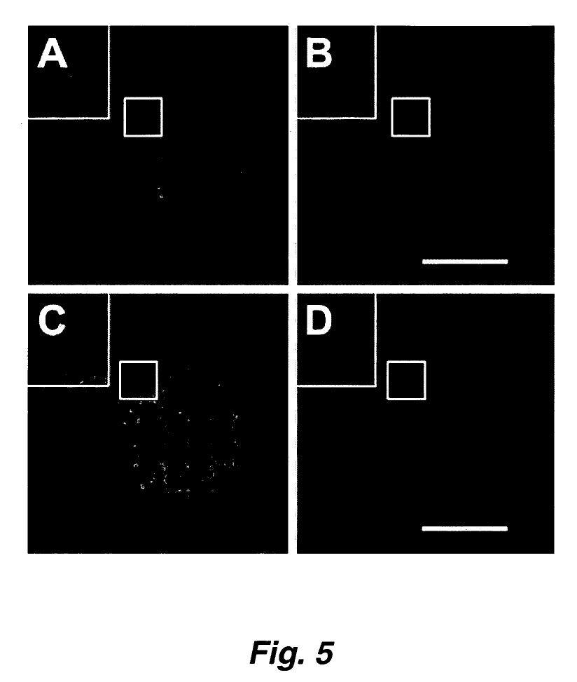

Though almost all neurons expressed both PKCε and β₂-AR (94.9%±1.8% (n=409) and 99.8% ±0.2% (n=407) of neurons, respectively) translocation of PKCε was detected in only 20-35% of neurons. As the culture system employed comprises a mixture of sensory neurons subserving different function, a subpopulation of neurons in which translocation occurs was identified using double staining with the marker for non-peptidergic nociceptive neurons, IB4. The vast majority of neurons translocating PKCε after β₂-AR stimulation showed strong 1B4 plasma membrane fluorescence signal (FIG. 5, 88.6±2.4% of PKCε translocating cells show IB4 staining (evaluated cultures: n=4, percentage of IB4 positive per PKCε translocating cells: 88.9%, 92.9%, 81.8%, 90.9%, total number of PKCε translocating cells: 45)).

Epac Activates PKCε to Induce Hyperalgesia

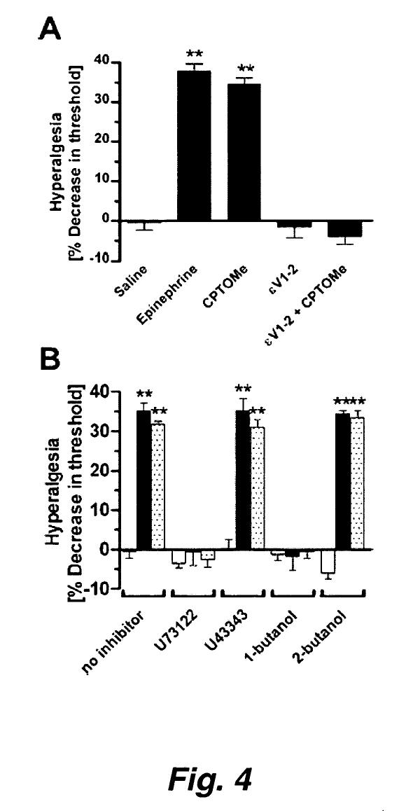

To determine if the signaling pathway upstream of PKCε in cultured nociceptive neurons applied also to PKCε signaling in nociceptor sensitization/hyperalgesia in vivo, behavioral experiments were performed. The same model as used to establish the role of PKCε in β-AR agonist-induced hyperalgesia was applied (Khasar et al., 1999b). The results showed that in vivo direct activation of Epac with CPTOMe robustly induces mechanical hyperalgesia (reduction of paw-withdrawal threshold by 37.7±1.9% after epinephrine (n=6), 34.6±1.6% after CPTOMe (n=12), increase of threshold by 0.4±1.8 after saline injection (n=6), FIG. 4A).

Next, the question of whether Epac mediates hyperalgesia through the activation of PKCε was examined by stimulating the onset of hyperalgesia with CPTOMe after the injection of the specific PKCε inhibitor εV1-2. As shown in FIG. 4A, εV1-2 inhibited hyperalgesia induced by CPTOMe (reduction of paw-withdrawal threshold by 34.6±1.6% after CPTOMe (n=12), increase by 1.6±2.7% after εV1-2 (n=6), and increas by 3.9±2.1% after CPTOMe into εV1-2 pretreated paws (n=6)). Therefore, the in vivo activation of Epac also leads to the activation of PKCε, which in turn leads to mechanical hyperalgesia.

Finally, to establish the role of PI—PLC and PLD as mediators of β₂-AR and Epac-induced hyperalgesia, the paws were preinjected with the respective inhibitors and their inactive controls, U73122/U73343 (PI—PLC) and 1-butanol/2-butanol (PLD), before the stimulation with either epinephrine or CPTOMe. Without stimulation neither inhibitor influenced the baseline sensitivity of the rats (n=6, U73122 −4.4%±1.3%, U73343 2.8%±1.9%, 1-butanol −0.0%±1.8%, 2-butanol −8%±1.7%, FIG. 4B). However, as seen in vitro, U73122 as well as 1-butanol inhibited the onset of CPTOMe-induced hyperalgesia (CPTOMe 31.7%±0.8% (n=12), U73122/CPTOMe −2.5%±2.0% (n=6), 1-butanol/CPTOMe −0.5%±1.8% (n=6)), while the respective negative controls, U73343 and 2-butanol, did not (U73343/CPTOMe 30.9%±1.9% (n=6), 2-butanol/CPTOMe 33.4±1.7% (n=6)). Thus, in vivo both, PI—PLC as well as PLD, could be established as essential mediators of mechanical hyperalgesia.

Discussion

Epac Mediates Crosstalk Ga/cAMP to PKCε

Extensive research on GPCR signaling has identified different pathways leading to the activation of PKC. The G-proteins α_{q} and βγ (Gudermann et al., 1997), or transactivation of growth factor receptors (Luttrell et al., 1997; Lee and Chao, 2001) but not the G-protein αₛ has been shown to lead to activation of phospholipases and thereby activation of PKCs. Recently, studies of nociceptor sensitization, in which AC was activated with forskolin, suggested that the G-protein as might activate PKC (Gold et al., 1998; Parada et al., 2005). Using the well-established activator of αₛ, cholera toxin, of AC, forskolin, and the cAMP analog, CPTOMe, this work provides proof of principle, that, indeed, the G-protein αₛ also mediates GPCR signaling toward activation of PKCε.

A mechanism connecting the cAMP and PKC signaling pathways had not been elucidated. In particular, the PKA inhibitor CMIQ, which blocks the ATP binding site of PKA (Lu et al., 1996), did not abolish β₂-AR-induced translocation of PKCε in DRG neurons, indicating the αₛ/cAMP second messenger signaling pathway to branch upstream of PKA before activating PKCε. The present work tested the hypothesis that the recently identified downstream mediator of cAMP, Epac (de Rooij et al., 1998; Kawasaki et al., 1998), could mediate this crosstalk. While Epac is known to activate MAPKs it is not known to activate PKCs. The Epac-specific cAMP analog CPTOMe was used show that activation of Epac induces PKCε translocation and therefore mediates the cAMP/PKC crosstalk in DRG neurons.

Epac Activates PKCε via PI—PLC and PLD

DAG generated by phospholipases can activate novel PKCs, such as PKCε. The β₂-AR has been shown in HEK293 cells to lead to activation of PLCε (Schmidt et al., 2001). The results obtained using the inhibitor U73122 indicated that the phospholipase involved in PKC& activation was of the same class as PLCε, namely a PI—PLC demonstrated its essential role in β₂-AR/Epac-induced activation of PKCε.

While PLCs often provide the first surge in DAG production, PLD can also contribute to a rise in DAG by production of phosphatidic acid (PA), which then can be converted into DAG (Nishizuka, 1992; Clapham and Neer, 1993). Surprisingly, however, PLD was found not only to be involved in, but to be necessary for, the translocation of PKCε. Of note in this regard, DAG as well as PA have recently been shown in RBL-2H3 cells to bind PKCε, at two different sites, and to both contribute to the attachment and activation of PKCε to the plasma membrane (Jose Lopez-Andreo et al., 2003). The present work shows that PLD is an essential component of PKCε translocation in nociceptors.

Epac Mediates PKCε-Dependent Mechanical Hyperalgesia

The present work investigated whether activation of Epac leads through PI—PLC and PLD also in vivo to PKCε-dependent mechanical hyperalgesia. PKCε dependent hyperalgesia has been shown to require the activity of PKCε in nociceptive neurons leading to modulation of the TTX—R sodium channel, which is one effector component central in pain (Khasar et al., 1999b; Parada et al., 2003b). Injection of modulators of PKCε into the hindpaw of rats has been proven to be a suitable way to modulate the activity of PKCε in nociceptive neurons and thereby to modulate PKCε-dependent hyperalgesia (Khasar et al., 1999b; Parada et al., 2003b). Injections of the Epac activator CPTOMe induces mechanical hyperalgesia to a similar extent as at the β₂-AR acting epinephrine. Epac activation leads also in vivo to activation of PKCε, as the use of the PKCε specific inhibitor, εV1-2, completely attenuated Epac activator-induced mechanical hyperalgesia, confirming the in vitro delineated signaling pathway.

The central role of both phospholipases, PI—PLC and PLD, in Epac/PKCε-induced hyperalgesia was confirmed in vivo. Inhibition of either PI—PLC with U73122 or inhibition of PLD with the inhibitor 1-butanol completely abolished Epac-mediated mechanical hyperalgesia, while the respective inhibitor controls, U73343 and 2-butanol, showed no effect. PI—PLC and PLD activity have been thereby introduced as new and essential components to β₂-AR-induced/PKCε-mediated mechanical hyperalgesia.

Epac Activates PKCε in IB4+ Nociceptors

The IB4-epitope is found on GDNF-dependent, small diameter, nociceptive neurons and marks about 30% of DRG-neurons (Molliver and Snider, 1997; Hunt and Mantyh, 2001). These neurons make up approximately 70% of neurons innervating the epidermis (Lu et al., 2001), project to lamina IIi of the spinal cord and are suggested to be involved in neuropathic pain (Molliver et al., 1997; Bennett et al., 1998; Boucher et al., 2000). While 1B4 has been used extensively for descriptive investigations of changes in protein expression in models of pain, little is known about the functional and mechanistic importance of these neurons. Translocation of PKCε was observed only in about 20-35% of cultured DRG neurons. Even though β₂-AR, Epac (data not shown), and PKCε are expressed in nearly all DRG neurons, translocation of PKCε was highly correlated with the expression of the IB4-epitope. The molecular basis for this specificity remains elusive and could be based on differential expression of additional signaling components not identified so far or on differential regulation of signaling components. The present observation corroborates IB4(+) neurons to be nociceptors and suggests a functional difference between IB4-positive and IB4-negative/peptidergic nociceptors. As the TTX—R sodium current is modulated by PKCε as well as other mechanisms (Khasar et al., 1999b) it will be interesting to investigate if also these additional mechanisms are present in IB4(+) neurons. While other molecules involved in nociception such as TRPV1 are expressed overlapping with different subpopulations of DRG neurons (Guo et al., 1999; Zwick et al., 2002), this work shows the first cellular mechanism restricted to the IB4(+) subtype of nociceptive neurons.

Epac and Other Signaling Pathways Mediating Hyperalgesia

In conclusion, by delineating a signaling pathway leading to the activation of PKCε in vitro and in vivo, this work presents a detailed analysis of an intracellular signaling pathway in sensory neurons underlying pain. The results elucidate thereby a novel mechanism of signal transduction, a crosstalk between the two ubiquitous signaling pathways cAMP and PKC, which are both used in a wide variety of systems as for example neuronal plasticity. The evidence indicates that Epac is a key element in this crosstalk. Importantly, this signaling pathway in DRG-neurons is restricted to the EB4(+) nociceptors, establishing a first mechanism specific to this subpopulation of nociceptors. Delineating the cascade leading to activation of PKCε this work introduces three new targets for treatment of pain, Epac, PI—PKC (e.g., PLCε), and PLD. Furthermore, this work provides proof of principle, that not only the G-proteins α₁ and βγ but also αₛ can activate PKC.

The results provide a powerful entry point to further investigate the intracellular signaling pathways also in hyperalgesia induced by other inflammatory mediators. Intriguingly, the literature of Epac and its downstream target, Rap1, suggests even further crosstalk also to other pathways important in nociceptor function such as PKA and MAPK (Khasar et al., 1999b; Aley et al., 2001; Stork and Schmitt, 2002), integrins (Bos et al., 2003; Rangarajan et al., 2003; Dina et al., 2004), and growth factor receptors (Boucher et al., 2000).

### REFERENCES

Aley K O, Messing R O, Mochly-Rosen D, Levine J D (2000) Chronic hypersensitivity for inflammatory nociceptor sensitization mediated by the epsilon isozyme of protein kinase C. JNeurosci 20:4680-4685.
Aley K O, Martin A, McMahon T, Mok J, Levine J D, Messing R O (2001) Nociceptor sensitization by extracellular signal-regulated kinases. JNeurosci 21:6933-6939.
Bennett D L, Michael G J, Ramachandran N, Munson J B, Averill S, Yan Q, McMahon S B, Priestley J V (1998) A distinct subgroup of small DRG cells express GDNF receptor components and GDNF is protective for these neurons after nerve injury. J Neurosci 18:3059-3072.
Bond R A, Leff P, Johnson T D, Milano C A, Rockman H A, McMinn T R, Apparsundaram S, Hyek M F, Kenakin T P, Allen L F, et al. (1995) Physiological effects of inverse agonists in transgenic mice with myocardial overexpression of the beta 2-adrenoceptor. Nature 374:272-276.
Bos J L, de Bruyn K, Enserink J, Kuiperij B, Rangarajan S, Rehmann H, Riedl J, de Rooij J, van Mansfeld F, Zwartkruis F (2003) The role of Rap1 in integrin-mediated cell adhesion. Biochem Soc Trans 31:83-86.
Boucher T J, Okuse K, Bennett D L, Munson J B, Wood J N, McMahon S B (2000) Potent analgesic effects of GDNF in neuropathic pain states. Science 290:124-127.
Cesare P, Dekker L V, Sardini A, Parker P J, McNaughton P A (1999) Specific involvement of PKC-epsilon in sensitization of the neuronal response to painful heat. Neuron 23:617-624.
Clapham D E, Neer E J (1993) New roles for G-protein beta gamma-dimers in transmembrane signalling. Nature 365:403-406.
de Rooij J, Zwartkruis F J, Verheijen M H, Cool R H, Nijman S M, Wittinghofer A, Bos J L (1998) Epac is a Rap1 guanine-nucleotide-exchange factor directly activated by cyclic AMP. Nature 396:474-477.
Dina O A, Chen X, Reichling D, Levine J D (2001) Role of protein kinase Cepsilon and protein kinase A in a model of paclitaxel-induced painful peripheral neuropathy in the rat. Neuroscience 108:507-515.
Dina O A, McCarter G C, de Coupade C, Levine J D (2003) Role of the sensory neuron cytoskeleton in second messenger signaling for inflammatory pain. Neuron 39:613-624.
Dina O A, Parada C A, Yeh J, Chen X, McCarter G C, Levine J D (2004) Integrin signaling in inflammatory and neuropathic pain in the rat. Eur J Neurosci 19:634-642.
Dina O A, Barletta J, Chen X, Mutero A, Martin A, Messing R O, Levine J D (2000) Key role for the epsilon isoform of protein kinase C in painful alcoholic neuropathy in the rat. J Neurosci 20:8614-8619.
Dorn G W, 2nd, Mochly-Rosen D (2002) Intracellular transport mechanisms of signal transducers. Annu Rev Physiol 64:407-429.
Enserink J M, Christensen A E, de Rooij J, van Triest M, Schwede F, Genieser H G, Doskeland S O, Blank J L, Bos J L (2002) A novel Epac-specific cAMP analogue demonstrates independent regulation of Rap1 and ERK. Nat Cell Biol 4:901-906.
Gold M S, Levine J D, Correa A M (1998) Modulation of TTX—R INa by PKC and PKA and their role in PGE2-induced sensitization of rat sensory neurons in vitro. J Neurosci 18:10345-10355.
Gudermann T, Schoneberg T, Schultz G (1997) Functional and structural complexity of signal transduction via G-protein-coupled receptors. Annu Rev Neurosci 20:399-427.
Guo A, Vulchanova L, Wang J, Li X, Elde R (1999) Immunocytochemical localization of the vanilloid receptor 1 (VR1): relationship to neuropeptides, the P2X3 purinoceptor and IB4 binding sites. Eur J Neurosci 11:946-958.
Hunt S P, Mantyh P W (2001) The molecular dynamics of pain control. NatRevNeurosci 2:83-91.
Johnson J A, Gray M O, Chen C H, Mochly-Rosen D (1996) A protein kinase C translocation inhibitor as an isozyme-selective antagonist of cardiac function. J Biol Chem 271:24962-24966.
Jose Lopez-Andreo M, Gomez-Fernandez J C, Corbalan-Garcia S (2003) The simultaneous production of phosphatidic acid and diacylglycerol is essential for the translocation of protein kinase Cepsilon to the plasma membrane in RBL-2H3 cells. Mol Biol Cell 14:4885-4895.
Joseph E K, Levine J D (2003a) Sexual dimorphism in the contribution of protein kinase C isoforms to nociception in the streptozotocin diabetic rat. Neuroscience 120:907-913.
Joseph E K, Levine J D (2003b) Sexual dimorphism for protein kinase c epsilon signaling in a rat model of vincristine-induced painful peripheral neuropathy. Neuroscience 119:831-838.
Kawasaki H, Springett G M, Mochizuki N, Toki S, Nakaya M, Matsuda M, Housman D E, Graybiel A M (1998) A family of cAMP-binding proteins that directly activate Rap1. Science 282:2275-2279.
Keiper M, Stope M B, Szatkowski D, Bohm A, Tysack K, Vom Dorp F, Saur O, Oude Weernink P A, Evellin S, Jakobs K H, Schmidt M (2004) Epac- and Ca2+-controlled activation of Ras and extracellular signal-regulated kinases by Gs-coupled receptors. Involvement of Rap2B-activated phospholipase C-epsilon. J Biol Chem.
Khasar S G, McCarter G, Levine J D (1999a) Epinephrine produces a beta-adrenergic receptor-mediated mechanical hyperalgesia and in vitro sensitization of rat nociceptors. J Neurophysiol 81:1104-1112.
Khasar S G, Ouseph A K, Chou B, Ho T, Green P G, Levine J D (1995) Is there more than one prostaglandin E receptor subtype mediating hyperalgesia in the rat hindpaw? Neuroscience 64:1161-1165.
Khasar S G, Lin Y H, Martin A, Dadgar J, McMahon T, Wang D, Hundle B, Aley K O, Isenberg W, McCarter G, Green P G, Hodge C W, Levine J D, Messing R O (1999b) A novel nociceptor signaling pathway revealed in protein kinase C epsilon mutant mice. Neuron 24:253-260.
Lee F S, Chao M V (2001) Activation of Trk neurotrophin receptors in the absence of neurotrophins. Proc Natl Acad Sci U S A 98:3555-3560.
Lu J, Zhou X F, Rush R A (2001) Small primary sensory neurons innervating epidermis and viscera display differential phenotype in the adult rat. Neurosci Res 41:355-363.
Lu Z X, Quazi N H, Deady L W, Polya G M (1996) Selective inhibition of cyclic AMP-dependent protein kinase by isoquinoline derivatives. Biol Chem Hoppe Seyler 377:373-384.
Luttrell L M, Della Rocca G J, van Biesen T, Luttrell D K, Lefkowitz R J (1997) Gbetagamma subunits mediate Src-dependent phosphorylation of the epidermal growth factor receptor. A scaffold for G protein-coupled receptor-mediated Ras activation. J Biol Chem 272:4637-4644.
Molliver D C, Snider W D (1997) Nerve growth factor receptor TrkA is down-regulated during postnatal development by a subset of dorsal root ganglion neurons. J Comp Neurol 381:428-438.
Molliver D C, Wright D E, Leitner M L, Parsadanian A S, Doster K, Wen D, Yan Q, Snider W D (1997) IB4-binding DRG neurons switch from NGF to GDNF dependence in early postnatal life. Neuron 19:849-861.
Nishizuka Y (1992) Intracellular signaling by hydrolysis of phospholipids and activation of protein kinase C. Science 258:607-614.
Numazaki M, Tominaga T, Toyooka H, Tominaga M (2002) Direct phosphorylation of capsaicin receptor VR1 by protein kinase Cepsilon and identification of two target serine residues. J Biol Chem 277:13375-13378.
Parada C A, Reichling D B, Levine J D (2005) Chronic hyperalgesic priming in the rat involves a novel interaction between cAMP and PKCvarepsilon second messenger pathways. Pain 113:185-190.
Parada C A, Yeh J J, Joseph E K, Levine J D (2003a) Tumor necrosis factor receptor type-1 in sensory neurons contributes to induction of chronic enhancement of inflammatory hyperalgesia in rat. Eur J Neurosci 17:1847-1852.
Parada C A, Yeh J J, Reichling D B, Levine J D (2003b) Transient attenuation of protein kinase Cepsilon can terminate a chronic hyperalgesic state in the rat. Neuroscience 120:219-226.
Parekh D B, Ziegler W, Parker P J (2000) Multiple pathways control protein kinase C phosphorylation. Embo J 19:496-503.
Penn R B, Parent J L, Pronin A N, Panettieri R A, Jr., Benovic J L (1999) Pharmacological inhibition of protein kinases in intact cells: antagonism of beta adrenergic receptor ligand binding by H-89 reveals limitations of usefulness. J Pharmacol Exp Ther 288:428-437.
Rangarajan S, Enserink J M, Kuiperij H B, de Rooij J, Price L S, Schwede F, Bos J L (2003) Cyclic AMP induces integrin-mediated cell adhesion through Epac and Rap1 upon stimulation of the beta 2-adrenergic receptor. J Cell Biol 160:487-493.
Rehmann H, Schwede F, Doskeland S O, Wittinghofer A, Bos J L (2003) Ligand-mediated activation of the cAMP-responsive guanine nucleotide exchange factor Epac. J Biol Chem 278:38548-38556.
Rizzo M, Romero G (2002) Pharmacological importance of phospholipase D and phosphatidic acid in the regulation of the mitogen-activated protein kinase cascade. Pharmacol Ther 94:35-50.
Schmidt M, Evellin S, Weernink P A, von Dorp F, Rehmann H, Lomasney J W, Jakobs K H (2001) A new phospholipase-C-calcium signalling pathway mediated by cyclic AMP and a Rap GTPase. Nat Cell Biol 3:1020-1024.
Schutze S, Potthoff K, Machleidt T, Berkovic D, Wiegmann K, Kronke M (1992) TNF activates NF-kappa B by phosphatidylcholine-specific phospholipase C-induced “acidic” sphingomyelin breakdown. Cell 71:765-776.
Slice L W, Taylor S S (1989) Expression of the catalytic subunit of cAMP-dependent protein kinase in Escherichia coli. J Biol Chem 264:20940-20946.
Stork P J, Schmitt J M (2002) Crosstalk between cAMP and MAP kinase signaling in the regulation of cell proliferation. Trends Cell Biol 12:258-266.
Sweitzer S M, Wong S M, Peters M C, Mochly-Rosen D, Yeomans D C, Kendig J J (2004) Protein kinase C {epsilon} and {gamma}: Involvement in formalin-induced nociception in neonatal rats. J Pharmacol Exp Ther.
Taiwo Y O, Levine J D (1989) Contribution of guanine nucleotide regulatory proteins to prostaglandin hyperalgesia in the rat. Brain Res 492:400-403.
Toullec D, Pianetti P, Coste H, Bellevergue P, Grand-Perret T, Ajakane M, Baudet V, Boissin P, Boursier E, Loriolle F, et al. (1991) The bisindolylmaleimide GF 109203X is a potent and selective inhibitor of protein kinase C. J Biol Chem 266:15771-15781.
Zwick M, Davis B M, Woodbury C J, Burkett J N, Koerber H R, Simpson J F, Albers K M (2002) Glial cell line-derived neurotrophic factor is a survival factor for isolectin B4-positive, but not vanilloid receptor 1-positive, neurons in the mouse. J Neurosci 22:4057-4065.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of reducing pain, said method comprising administering to a subject in need thereof, an effective amount of an inhibitor selected from the group consisting of an Epac inhibitor, a phospholipase C-epsilon (PLCε) inhibitor, and a phospholipase D (PLD) inhibitor.

2. The method of claim 1, wherein said administration results in the subject having decreased hyperalgesia.

3. The method of claim 2, wherein said administration has no significant effect on nociception in the subject.

4. The method of claim 1, wherein the subject suffers from inflammatory pain.

5. The method of claim 4, wherein the inflammatory pain is acute.

6. The method of claim 4, wherein the inflammatory pain is chronic.

7. The method of claim 4, wherein the inflammatory pain is due to a condition selected from the group consisting of: sunburn, arthritis, colitis, carditis, dermatitis, myositis, neuritis, mucositis, urethritis, cystitis, gastritis, pneumonitis,and collagen vascular disease.

8. The method of claim 1, wherein the subject suffers from neuropathic pain.

9. The method of claim 8, wherein the neuropathic pain is acute.

10. The method of claim 8, wherein the neuropathic pain is chronic.

11. The method of claim 8, wherein the neuropathic pain is due to a condition selected from the group consisting of: causalgia, diabetes, collagen vascular disease, trigeminal neuralgia, spinal cord injury, brain stem injury, thalamic pain syndrome, complex regional pain syndrome type I/reflex sympathetic dystrophy, Fabry's syndrome, small fiber neuropathy, cancer, cancer chemotherapy, chronic alcoholism, stroke, abscess, demyelinating disease, viral infection, anti-viral therapy, AIDS, and AIDS therapy.

12. The method of claim 8, wherein said neuropathic pain is due to an agent selected from the group consisting of: trauma, surgery, amputation, toxin, and chemotherapy.

13. The method of claim 1, wherein the subject suffers from a generalized pain disorder.

14. The method of claim 13, wherein the generalized pain disorder is selected from the group consisting of fibromyalgia, irritable bowel syndrome, and a temporomandibular disorder.

15. The method of claim 1, wherein the inhibitor is an Epac inhibitor.

16. The method of claim 15, wherein the Epac inhibitor acts directly on Epac.

17. The method of claim 15, said method additionally comprising: administering to the subject an analgesic agent that acts by a different mechanism than the Epac inhibitor.

18. The method of claim 1, wherein the inhibitor is a PLCε inhibitor.

19. The method of claim 15, wherein the PLCε inhibitor acts directly on PLCε.

20. The method of claim 15, said method additionally comprising: administering to the subject an analgesic agent that acts by a different mechanism than the PLCε inhibitor.

21. The method of claim 1, wherein the inhibitor is a PLD inhibitor.

22. The method of claim 17, wherein the PLD inhibitor acts directly on PLD.

23. The method of claim 21, wherein the PLD inhibitor is a selective PLD inhibitor.

24. The method of claim 21, said method additionally comprising: administering to the subject an analgesic agent that acts by a different mechanism than the PLD inhibitor.

25. The method of claim 1, wherein said method also comprises administering an agent selected from the group consisting of: an inhibitor of protein kinase A (PKA), an inhibitor of cAMP, a nonsteroidal anti-inflammatory drug, a prostaglandin synthesis inhibitor, a local anesthetic, an anticonvulsant, an antidepressant, an opioid receptor agonist, and a neuroleptic.

26. A pharmaceutical composition comprising:
(a) an inhibitor selected from the group consisting of an Epac inhibitor, a PLCε inhibitor, and a PLD inhibitor; and
(b) an analgesic agent that acts by a different mechanism than said inhibitor.

27. A pharmaceutical composition comprising:
(a) an inhibitor selected from the group consisting of an Epac inhibitor, a PLCε inhibitor, and a PLD inhibitor; and
(b) an agent selected from the group consisting of: an inhibitor of protein kinase A (PKA), an inhibitor of cAMP, a nonsteroidal anti-inflammatory drug, prostaglandin synthesis inhibitor, a local anesthetic, an anticonvulsant, an antidepressant, an opioid receptor agonist, and a neuroleptic.

28. (canceled)

29. (canceled)

30. (canceled)

31. A method of prescreening for an agent that can reduce pain in a subject, the method comprising:
(a) contacting a test agent with a polypeptide selected from the group consisting of Epac, PLCε, and PLD;
(b) determining whether the test agent specifically binds to the polypeptide; and
(c) if the test agent specifically binds to the polypeptide, selecting the test agent as a potential analgesic.

32. A method of prescreening for an agent that can reduce pain in a subject, the method comprising:
(a) contacting a test agent with a polynucleotide encoding a polypeptide selected from the group consisting of Epac, PLCε, and PLD;
(b) determining whether the test agent specifically binds to the polynucleotide; and
(c) if the test agent specifically binds to the polynucleotide, selecting the test agent as a potential analgesic.

33. (canceled)

34. (canceled)

35. (canceled)

36. (canceled)

37. (canceled)

38. A method of screening for an agent that can reduce pain in a subject, the method comprising:
(a) contacting a test agent with polypeptide selected from the group consisting of Epac, PLCε, and PLD;
(b) determining whether the test agent inhibits the polypeptide; and
(c) if the test agent inhibits the polypeptide, selecting the test agent as a potential analgesic.

39. (canceled)

40. (canceled)

41. (canceled)

42. (canceled)

43. (canceled)

44. (canceled)

45. (canceled)

46. (canceled)

47. (canceled)

48. A method of screening for an agent that that can reduce pain in a subject, the method comprising:
(a) selecting an inhibitor selected from the group consisting of an Epac inhibitor, a PLCε inhibitor, and a PLD inhibitor as a test agent; and
(b) measuring the ability of the selected test agent to reduce pain in an animal model.

49. A kit comprising:
(a) an inhibitor selected from the group consisting of an Epac inhibitor, a PLCε inhibitor, and a PLD inhibitor in a pharmaceutically acceptable carrier;
(b) instructions for carrying out the method of claim 1.


SEQUENCE LISTING (1 sequence)

SEQ ID NO: 1 - RNA, 14 nt, Artificial
  sequence requirement for hairpin ribozyme
  misc_feature (1)..(3): n = g, u, c, or a
  misc_feature (4)..(4): b = g, c, or u
  misc_feature (5)..(5): n = g, u, c, or a
  misc_feature (9)..(14): n = g, u, c, or a
nnnbngucnnnnnn